(19) United States
(12) Patent Application Publication
(10) Pub. No.: US 2012/0045509 A1
(43) Pub. Date: Feb. 23, 2012

(54) MODIFIED RELEASE COMPOSITIONS FOR DPP-IV INHIBITORS

(76) Inventors: Bernd Michael Loeffler, Zug (CH); Alexander MacDonald, Chesham (GB); Cynthia Rocha, Torino (IT); Eric Worth, Bedfordshire (GB)

(21) Appl. No.: 13/282,889

(22) Filed: Oct. 27, 2011

Related U.S. Application Data
(63) Continuation of application No. 11/499,587, filed on Aug. 4, 2006, now abandoned.

(30) Foreign Application Priority Data
Aug. 11, 2005 (EP) 05107393.0

(51) Int. Cl. A61K 9/48; A61K 9/28; A61K 9/20; A61K 9/24; C07D 413/12; C07D 401/14; C07D 455/06; C07D 207/16; C07D 209/52; C07D 487/04; A61K 31/422; A61K 31/4439; A61K 31/4745; A61K 31/40; A61K 31/403; A61K 31/4985; A61P 3/10; A61P 3/08; A61K 9/00

(52) U.S. Cl. 424/463; 424/400; 424/474; 424/451; 424/464; 424/472; 548/236; 546/275.1; 546/95; 548/540; 548/515; 544/350; 514/374; 514/341; 514/294; 514/423; 514/412; 514/249

(57) ABSTRACT

The present invention refers to pharmaceutical composition comprising a DPP-IV inhibitor.

### PRIORITY TO RELATED APPLICATIONS

This application is a continuation of U.S. application Ser. No. 11/499,587, filed Aug. 4, 2006, now Pending, which claims the benefit of European Application No. 05107393.0, filed Aug. 11, 2005, which is hereby, incorporated by reference in its entirety.

### FIELD OF THE INVENTION

The present invention relates to new pharmaceutical compositions comprising a DPP-IV inhibitor.

All documents cited or relied upon below are expressly incorporated herein by reference.

### BACKGROUND

The enzyme dipeptidyl peptidase IV (EC.3.4.14.5, abbreviated in the following as DPP-IV) is involved in the regulation of the activities of several hormones. In particular DPP-IV degrades efficiently and rapidly glucagon like peptide 1 (GLP-1), one of the most potent stimulators of insulin production and secretion. Inhibiting DPP-IV would potentiate the effect of endogenous GLP-1, leading to higher plasma insulin concentrations. In patients suffering from impaired glucose tolerance and type 2 diabetes mellitus, the resultant higher plasma insulin concentration would reduce the dangerous hyperglycaemia and accordingly reduce the risk of late diabetic complications. Consequently, DPP-IV inhibitors have been suggested as drug candidates for the treatment of impaired glucose tolerance and diabetes, particularly type 2 diabetes mellitus (e.g. Vilhauer, WO98/19998). Other related state of the art can be found in WO 99/38501, DE 19616486, DE 19834591, WO 01/40180, WO 01/55105, U.S. Pat. No. 6,110,949, WO 00/34241

There are three recognized types of diabetes mellitus. Type I diabetes or insulin dependent diabetes mellitus (IDDM) is typically of juvenile onset; ketosis develops early in life with much more severe symptoms and has a near-certain prospect of later vascular involvement. Control of Type I diabetes is difficult and requires exogenous insulin administration. Type II diabetes or non-insulin dependent diabetes mellitus (NIDDM) is ketosis-resistant, generally develops later in life, is milder and has a more gradual onset. Type III diabetes is malnutrition-related diabetes.

Type II diabetes is a condition that poses a major threat to the health of the citizens of the western world. Type II diabetes accounts for over 85% of diabetes incidence worldwide and about 160 million people are suffering from type II diabetes. The incidence is expected to increase considerably within the next decades, especially in developing countries. Type II diabetes is associated with morbidity and premature mortality resulting from serious complications, e.g. cardiovascular disease (Weir, G. C., Leahy, J. L., (1994), Pathogenesis of non-insulin dependent (Type II) diabetes mellitus. Joslin's Diabetes Mellitus 13th Ed. (Kahn, C. R., Weir, G. C., Eds.), Lea & Febiger, Malvern, Pa., pp. 240-264). Type II diabetes is characterized by both fasting and post-prandial hyperglycemia resulting from abnormalities in insulin secretion and insulin action, i.e. insulin resistance (Weir, G. C. et al. vide supra). In the insulin resistant state, the peripheral tissues and the liver exhibit a reduced sensitivity to insulin whereby the stimulation of glucose uptake into muscle and fat cells by insulin is blunted and the suppression of hepatic glucose output by insulin is incomplete.

The hyperglycemia in patients suffering from type II diabetes can usually be initially treated by dieting, but eventually most type II diabetes patients have to take oral antidiabetic agents and/or insulin injections to normalize their blood glucose levels. The introduction of orally effective hypoglycemic agents was an important development in the treatment of hyperglycemia by lowering blood glucose levels. Currently, the most widely used oral antidiabetic agents are the sulfonylureas, which act by increasing the secretion of insulin from the pancreas (Lebovitz, H. E., (1994) Oral antidiabetic agents. Joslin's Diabetes Mellitus 13th Ed. (Kahn, C. R., Weir G. C., Eds.), Lea & Febiger, Malvern, Pa., pp. 508-529), the biguanides (e.g. metformin) which act on the liver and periphery by unknown mechanisms (Bailey, C. J., Path, M. R. C., Turner R. C. (1996) N. Engl. J. Med. 334: 574) and the thiazolidinediones (e.g. rosiglitazone/Avandia®) which enhance the effects of insulin at peripheral target sites (Plosker, G. L., Faulds, D., (1999) Drugs, 57(3), 409-438).

These existing therapies which comprise a wide variety of biguanide, sulfonylurea and thiazolidinedione derivatives have been used clinically as hypoglycemic agents. However, all three classes of compound have side effects. The biguanides, for example metformin, are unspecific and in certain cases have been associated with lactic acidosis, and need to be given over a longer period of time, i.e. they are not suitable for acute administration (Bailey et al., vide supra). The sulfonylureas, though having good hypoglycemic activity, require great care during use because they frequently cause serious hypoglycemia and are most effective over a period of circa ten years. The thiazolidinediones may cause weight gain following chronic administration (Plosker and Faulds, vide supra) and troglitazone has been associated with the occurrence of serious hepatic dysfunction.

Concerning the use of DPP-IV inhibitors for the treatment of diabetes and related diseases, there is still the need to increase the efficacy of the administration and to decrease potential side effects. It has now unexpectedly been found that the new pharmaceutical compositions according to the present invention exhibit advantages over other formulations comprising DPP-IV inhibitors already known in the art.

### SUMMARY OF THE INVENTION

In one embodiment of the present invention, provided is a pharmaceutical composition comprising a therapeutically effective amount of a DPP-IV inhibitor, wherein the DPP-IV inhibitor is released in the lower gastrointestinal tract.

In another embodiment of the present invention, provided is a method for the treatment of diseases associated with elevated blood glucose levels, comprising the step of administering a therapeutically effective amount of a pharmaceutical composition herein described to a human being or animal in need thereof.

### DETAILED DESCRIPTION

Until recently, it was generally assumed that a successful and potent DPP-IV inhibitor has to block as much as possible the plasmatic activity of the soluble form of DPP-IV. The plasma was assumed to be the important site of action. Consequently, the capability of a DPP-IV inhibitor to inhibit as completely as possible and as long as possible the plasma DPP-IV was assumed to be essential (Ahren, B. et al. Inhibition of Dipeptidyl Peptidase IV Improves Metabolic Control Over a 4-Week Study Period in Type 2 Diabetes. Diabetes Care 25, 869-875 (2002)). It has now surprisingly been found that the plasma level of a DPP-IV inhibitor is of less importance than previously assumed and that a site specific delivery of a DPP-IV inhibitor results in a largely increased efficacy and in a different type of antidiabetic activity with improved pharmacology. In particular, it was found that a site specific delivery in the lower gastrointestinal tract, particularly the ileum, is most desirable in humans. The present invention therefore is concerned with pharmaceutical compositions comprising a DPP-IV inhibitor, characterized in that the DPP-IV inhibitor is released in the lower gastrointestinal tract.

Unless otherwise indicated, the following definitions are set forth to illustrate and define the meaning and scope of the various terms used to describe the invention herein.

The term “lower gastrointestinal tract” refers to the jejunum, ileum, caecum and ascending colon, preferably the ileum, caecum and ascending colon.

The term “upper gut” refers to the stomach including the pylorus, pyloral sphincta and duodenal bulb.

The term “DPP-IV inhibitor” refers to a compound that exhibits inhibitory activity on the enzyme dipeptidyl peptidase IV. Such inhibitory activity can be characterized by the IC₅₀ value. A DPP-IV inhibitor preferably exhibits an IC₅₀ value below 10 μM, preferably below 1 μM. IC₅₀ values of DPP-IV inhibitors are usually above 0.01 nM, preferably above 0.1 nM.

The term “IC₅₀ value” refers to the concentration of inhibitor, particularly DPP-IV inhibitor, at which DPP-IV activity is inhibited by 50%.

In detail, the present invention is concerned with a pharmaceutical composition comprising a DPP-IV inhibitor, characterized in that the DPP-IV inhibitor is released in the lower gastrointestinal tract, preferably the ileum. Such compositions are preferably orally administrable.

A preferred embodiment of the present invention relates to a pharmaceutical composition as defined above, wherein the DPP-IV inhibitor is released at a pH above 7.0, preferably above 7.2.

The pharmaceutical composition of the present invention preferably comprises a coating. Such a coating is used to achieve the release of the DPP-IV inhibitor in the lower gastrointestinal tract or ileum, preferably the ileum. The release characteristics of the coating are chosen adequately, in order to achieve the release of the DPP-IV inhibitor in the lower gastrointestinal tract or ileum. Appropriate coatings dissolve at the desired pH, e.g. at pH 7.0. Once the coating is dissolved, the DPP-IV inhibitor is released from the composition and can be absorbed. Preferably, the coating is dissolved and at least 90% of the DPP-IV inhibitor is released within 120 minutes after exposure to the desired pH. Preferably, the coating is dissolved after 30 to 60 minutes and the DPP-IV inhibitor is thereafter preferably completely released within 60 minutes. The release of the DPP-IV inhibitor can be measured, e.g. in vitro by methods commonly known to the person skilled in the art.

Examples of suitable coatings are e.g. copolymers of Methacrylic acid, Methyl methacrylate, Ethylmethacyrlate, Methyacrylate and mixtures thereof. Such coatings are commercially available, e.g. as “Eudragit S”, “Eudragit L”, “Eudragit RS”, “Eudragit RL” and “Eudragit FS”, preferably “Eudragit S” and “Eudragit RS”, more preferably “Eudragit S”.

Another preferred embodiment of the present invention is a pharmaceutical composition as defined above, wherein the composition is a tablet or a capsule. Such tablets or capsules can preferably comprise a coating. Another embodiment of the present invention refers to tablets or capsules as defined above, wherein the tablet or capsule comprises coated pellets. Such tablets or capsules individually constitute separate embodiments of the present invention.

A preferred pharmaceutical composition as defined above is one, wherein at least 80%, preferably at least 90%, more preferably at least 95% of the DPP-IV inhibitor is released in the lower gastrointestinal tract, particularly the ileum. Preferably less than 10%, more preferably none, of the DPP-IV inhibitor is released prior to the lower gastrointestinal tract or ileum. Preferably less than 10%, more preferably none, of the DPP-IV inhibitor is released in the duodenum.

In the pharmaceutical composition as defined above, it is preferred that the DPP-IV inhibitor is released with a delay of 15 minutes, more preferably 30 to 60 minutes, at pH 7.0, more preferably pH 7.2.

A pharmaceutical composition as defined above, comprising 10 to 1000 mg of the DPP-IV inhibitor, is preferred, particularly a pharmaceutical composition comprising 10 to 400 mg of the DPP-IV inhibitor, more preferably 100 to 400 mg.

A preferred embodiment of the present invention refers to a pharmaceutical composition as defined above, wherein the DPP-IV inhibitor exhibits a biological activity characterized by an IC₅₀ value below 10 μM, more preferably below 1 μM. Preferably, the DPP-IV inhibitor is further characterized by an IC₅₀ value above 0.01 nM, preferably above 0.1 nM. IC₅₀ values can be determined by methods well known to the person skilled in the art, e.g. by the method described in this document.

A number of DPP-IV inhibitors have been reported in recent years for example in the following documents:

WO9946272, WO9819998, WO9308259, WO9116339, WO2005058901, WO2005056541, WO2005051950, WO2005051949, WO2005047297, WO2005044195, WO2005042488, WO2005040095, WO2005037828, WO2005037779, WO2005033106, WO2005033099, WO2005026148, WO2005025554, WO2005023762, WO2005021550, WO2005021536, WO2005012312, WO2005012308, WO2005011581, WO2005003135, WO2004112701, WO2004111041, WO2004110436, WO2004108730, WO2004103993, WO2004103276, WO2004101514, WO2004099185, WO2004099134, WO2004096806, WO2004092128, WO2004089362, WO2004087053, WO2004076434, WO2004076433, WO2004071454, WO2004069162, WO2004067509, WO2004064778, WO2004058266, WO2004052850, WO2004050658, WO2004050656, WO2004050022, WO2004048379, WO2004048352, WO2004046106, WO2004043940, WO2004041795, WO2004037181, WO2004037169, WO2004033455, WO2004032836, WO2004026822, WO2004018468, WO2004014860, WO2004007468, WO2004007446, WO03101958, WO03101449, WO03095425, WO03084940, WO03072556, WO03057144, WO03024965, WO03015775, WO03004498, WO03004496, WO03002595, WO03002593, WO03002553, WO03002531, WO03002530, WO03000181, WO03000180, WO02083128, WO02076450, WO0202560, WO0196295, WO0168603, WO0155105, WO0134594, WO0034241, U.S. Pat. No. 6,617,340, U.S. Pat. No. 6,548,481, U.S. Pat. No. 6,172,081, U.S. Pat. No. 6,124,305, U.S. Pat. No. 6,110,949, U.S. Pat. No. 6,107,317, U.S. Pat. No. 6,011,155, U.S. Pat. No. 5,939,560, U.S. Pat. No. 5,543,396, US2005153973, US2005143377, US2005137224, US2005131019, US2005130981, US2005107390, US2005107308, US2005065144, US2005043299, US2005043292, US2005038020, US2005004205, US2004259903, US2004259902, US2004259843, US2004235752, US2004229848, US2004209891, US2004152745, US2004121964, US2004116328, US2004082607, US2004082497, US2003216450, US2003216382, US2003195188, US2003148961, US2003130281, US2003096857, US2003087950, US2003078247, US2001020006, JP2005170792, JP2004244412, JP2004026820, JP2004002368, JP2004002367, JP2003327532, JP2003300977, JP2002265439, EP1541551, EP1541148, EP1541143, EP1535907, EP1535906, EP1506967, EP1489088, EP1457494, EP1426366, EP1354882, EP1338595, EP1333025, EP1323710, EP1308439, EP1258480, EP1184388, EP1043328, DE10327439, DE10254304, DE10251927, DE10238477, DE10238470, DE10109021, DD296075, AU2003261487.

Suitable DPP-IV inhibitors include but are not limited to those described in the above-referenced documents.

Reference herein to a DPP-IV inhibitors includes a reference to pharmaceutically acceptable salt, esters and derivatives thereof.

In the pharmaceutical compositions according to the present invention, the DPP-IV inhibitor can preferably be a compound of formula (I)

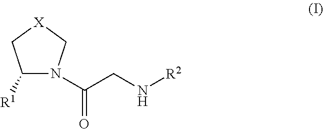

wherein

### R¹ is H or CN,

R² is —C(R³,R⁴)—(CH₂)ₙ—R⁵, —C(R³,R⁴)—CH₂—NH—R⁶, —C(R³,R⁴)—CH₂—O—R⁷; or
tetralinyl, tetrahydroquinolinyl or tetrahydroisoquinolinyl, which tetralinyl, tetrahydroquinolinyl or tetrahydroisoquinolinyl group can optionally be substituted with 1 to 3 substituents independently selected from the group consisting of lower-alkyl, lower-alkoxy, halogen, CN, and CF₃,
R³ is hydrogen, lower-alkyl, benzyl, hydroxybenzyl or indolylmethylene,
R⁴ is hydrogen or lower-alkyl, or
R³ and R⁴ are bonded to each other to form a ring together with the carbon atom to which they are attached and —R³—R⁴— is —(CH₂)₂₋₅—,
R⁵ is 5-membered heteroaryl, bi- or tricyclic heterocyclyl, or aminophenyl; optionally substituted with 1 to 3 substituents independently selected from the group consisting of lower-alkyl, lower-alkoxy, halogen, CN, CF₃, trifluoroacetyl, thiophenyl, phenyl, heteroaryl and monocyclic heterocyclyl, which phenyl, heteroaryl or monocyclic heterocyclyl can optionally be substituted with 1 to 3 substituents independently selected from the group consisting of lower-alkyl, lower-alkoxy, benzyloxy, halogen, CF₃, CF₃—O, CN and NH—CO-lower-alkyl,
R⁶ is a) pyridinyl or pyrimidinyl, which is substituted with 1 to 3 substituents independently selected from the group consisting of aryl and heteroaryl, which aryl or heteroaryl group can optionally be substituted with 1 to 3 substituents independently selected from the group consisting of lower-alkyl, lower-alkoxy, halogen, CN, and CF₃,

or b) 5-membered heteroaryl or bi- or tricyclic heterocyclyl, which 5-membered heteroaryl or bi- or tricyclic heterocyclyl can optionally be substituted with 1 to 3 substituents independently selected from the group consisting of lower-alkyl, carbonyl, aryl and heteroaryl, which aryl or heteroaryl group can optionally be substituted with 1 to 3 substituents independently selected from the group consisting of lower-alkyl, lower-alkoxy, halogen, CN, and CF₃, and which carbonyl group can optionally be substituted with lower-alkyl, lower-alkoxy, halogen, CN, CF₃, aryl, or heteroaryl, which aryl or heteroaryl group can optionally be substituted with 1 to 3 substituents independently selected from the group consisting of lower-alkyl, lower-alkoxy, halogen, CN, and CF₃,

R⁷ is aminophenyl, naphthyl or quinolinyl, optionally substituted with 1 to 3 substituents independently selected from the group consisting of lower-alkyl, lower-alkoxy, halogen, CN and CF₃,

### X is C(R⁸,R⁹) or S,

R⁸ and R⁹ independently from each other are H or lower-alkyl,
n is 0, 1 or 2,
and pharmaceutically acceptable salts thereof.

DPP-IV inhibitors according to formula (I) preferably include those selected from the group consisting of
(2S)-1-[((1R/S)-1,2,3,4-Tetrahydro-naphthalen-1-ylamino)-acetyl]-pyrrolidine-2-carbonitrile,
(2S)-1-[((2R/S)-6-Methoxy-1,2,3,4-tetrahydro-naphthalen-2-ylamino)-acetyl]-pyrrolidine-2-carbonitrile,
(2S)-1-[((2R/S)-1,2,3,4-Tetrahydro-naphthalen-1-ylamino)-acetyl]-pyrrolidine-2-carbonitrile,
(2S)-1-{[(1S)-2-(5-Methoxy-2-methyl-indol-1-yl)-1-methyl-ethylamino]-acetyl}-pyrrolidine-2-carbonitrile,
(2S)-1-{[(1S)-2-(5-cyano-indol-1-yl)-1-methyl-ethylamino]-acetyl}-pyrrolidine-2-carbonitrile,
(2S)-1-{[(1S)-1-Methyl-2-(2-methyl-indol-1-yl)-ethylamino]-acetyl}-pyrrolidine-2-carbonitrile,
(2S)-1-{[(1S)-2-(2,3-Dimethyl-indol-1-yl)-1-methyl-ethylamino]-acetyl}-pyrrolidine-2-carbonitrile,
(2S)-1-{[(1S)-1-Methyl-2-(3-methyl-indol-1-yl)-ethylamino]-acetyl}-pyrrolidine-2-carbonitrile,
(2S)-1-{[(1S)-2-(5-Brom-indol-1-yl)-1-methyl-ethylamino]-acetyl}-pyrrolidine-2-carbonitrile,
(2S)-1-{[2-(5-Brom-2,3-dihydro-indol-1-yl)-ethylamino]-acetyl}-pyrrolidine-2-carbonitrile,
(2S)-1-{[(1S)-2-(7-aza-indol-1-yl)-1-methyl-ethylamino]-acetyl}-pyrrolidine-2-carbonitrile,
(2S)-1-{[(1S)-2-(2-aza-indol-1-yl)-1-methyl-ethylamino]-acetyl}-pyrrolidine-2-carbonitrile,
(2S)-1-{[(1S)-1-Methyl-2-(5-phenyl-2,3-dihydro-indol-1-yl)-ethylamino]-acetyl}-pyrrolidine-2-carbonitrile,
(2S)-1-{[(1S)-2-(5-cyano-2-methyl-indol-1-yl)-1-methyl-ethylamino]-acetyl}-pyrrolidine-2-carbonitrile,
(2S)-1-{[(1S)-1-Methyl-2-(2-phenyl-indol-1-yl)-ethylamino]-acetyl}-pyrrolidine-2-carbonitrile,
(2S)-1-[((1S)-2-Carbazol-9-yl-1-methyl-ethylamino)-acetyl]-pyrrolidine-2-carbonitrile,
(2S)-1-{[(1S)-2-(6-Brom-indol-1-yl)-1-methyl-ethylamino]-acetyl}-pyrrolidine-2-carbonitrile,
(2S)-1-{[(1S)-1-Methyl-2-(7-methyl-indol-1-yl)-ethylamino]-acetyl}-pyrrolidine-2-carbonitrile,
(2S)-1-{[(1S)-2-(7-Brom-indol-1-yl)-1-methyl-ethylamino]-acetyl}-pyrrolidine-2-carbonitrile,
(2S)-1-{[2-(4-Chlor-indol-1-yl)-ethylamino]-acetyl}-pyrrolidine-2-carbonitrile,
(2S)-1-{[2-(5-Methoxy-2-methyl-indol-1-yl)-ethylamino]-acetyl}-pyrrolidine-2-carbonitrile,
(2S)-1-{[(1S)-2-(5,6-Dimethoxy-indol-1-yl)-1-methyl-ethylamino]-acetyl}-pyrrolidine-2-carbonitrile,
(2S)-1-{[(1S)-2-(5,6-Dimethoxy-3-trifluoroacetyl-indol-1-yl)-1-methyl-ethylamino]-acetyl}-pyrrolidine-2-carbonitrile,
(2S)-1-({(1S)-2-[6-(4-Methoxy-phenyl)-2,3-dihydro-indole-1-yl]-1-methyl-ethylamino}-acetyl)-pyrrolidine-2-carbonitrile,
(2S)-1-{[(1S)-1-Methyl-2-(naphthalen-2-yloxy)-ethylamino]-acetyl}-pyrrolidine-2-carbonitrile,
(2S)-1-{[2-(quinolin-6-yloxy)-ethylamino]-acetyl}-pyrrolidine-2-carbonitrile,
(2S)-1-{[2-(3-N,N-dimethylamino-phenoxy)-ethylamino]-acetyl}-pyrrolidine-2-carbonitrile,
(2S)-1-{[(1S)-2-(4-N,N-dimethylamino-phenyl)-1-methyl-ethylamino]-acetyl}-pyrrolidine-2-carbonitrile,
(2S)-1-{[(1R)-2-(4-N,N-dimethylamino-phenyl)-1-methyl-ethylamino]-acetyl}-pyrrolidine-2-carbonitrile,
(2S)-1-{[(1S)-2-(3-N,N-dimethylamino-phenyl)-1-methyl-ethylamino]-acetyl}-pyrrolidine-2-carbonitrile,
(2S)-1-{[2-(5-Methyl-2-phenyl-oxazol-4-yl)-ethylamino]-acetyl}-pyrrolidine-2-carbonitrile,
(2S)-1-({2-[2-(4-Fluoro-phenyl)-5-methyl-oxazol-4-yl]-ethylamino}-acetyl)-pyrrolidine-2-carbonitrile,
(2S)-1-({2-[2-(4-Benzyloxy-phenyl)-5-methyl-oxazol-4-yl]-ethylamino}-acetyl)-pyrrolidine-2-carbonitrile,
(2S)-1-({2-[2-(2-Ethoxy-4-fluoro-phenyl)-5-methyl-oxazol-4-yl]-ethylamino}-acetyl)-pyrrolidine-2-carbonitrile,
(2S)-1-({2-[2-(4-Chloro-phenyl)-5-methyl-oxazol-4-yl]-ethylamino}-acetyl)-pyrrolidine-2-carbonitrile,
(2S)-1-({2-[5-(4-Methoxy-phenyl)-pyridin-2-ylamino]-1,1-dimethyl-ethylamino}-acetyl)-pyrrolidine-2-carbonitrile,
(2S)-1-({2-[5-(4-Methoxy-phenyl)-pyridin-2-ylamino]-ethylamino}-acetyl)-pyrrolidine-2-carbonitrile,
1-({2-[5-(4-Methoxy-phenyl)-pyridin-2-ylamino]-ethylamino}-acetyl)-pyrrolidine,
(2S)-1-({2-[5-(3-Methoxy-phenyl)-pyridin-2-ylamino]-ethylamino}-acetyl)-pyrrolidine-2-carbonitrile,
(2S)-1-({2-[5-(2-Methoxy-phenyl)-pyridin-2-ylamino]-ethylamino}-acetyl)-pyrrolidine-2-carbonitrile,
(2S)-1-({2-[3-(4-Cyano-phenyl)-pyridin-2-ylamino]-ethylamino}-acetyl)-pyrrolidine-2-carbonitrile,
(2S)-1-({2-[5-Phenyl-pyridin-2-ylamino]-ethylamino}-acetyl)-pyrrolidine-2-carbonitrile,
1-({2-[5-Phenyl-pyridin-2-ylamino]-ethylamino}-acetyl)-pyrrolidine,
(2S)-1-({2-[6-Phenyl-pyridin-2-ylamino]-ethylamino}-acetyl)-pyrrolidine-2-carbonitrile,
(2S)-1-({2-[5-(5-Methyl-[1,3,4]oxadiazol-2-yl)-pyridin-2-ylamino]-ethylamino}-acetyl)-pyrrolidine-2-carbonitrile,
(2S)-1-({2-[3-(5-Methyl-[1,3,4]oxadiazol-2-yl)-pyridin-2-ylamino]-ethylamino}-acetyl)-pyrrolidine-2-carbonitrile,
(2S)-1-{[2-(4,5-Dimethyl-thiazol-2-ylamino)-ethylamino]-acetyl}-pyrrolidine-2-carbonitrile,
(2S)-1-({2-[4-(4-Cyano-phenyl)-thiazol-2-ylamino]-ethylamino}-acetyl)-pyrrolidine-2-carbonitrile,
1-({2-[4-(4-Cyano-phenyl)-thiazol-2-ylamino]-ethylamino}-acetyl)-pyrrolidine,
(2S)-1-({2-[4-(4-Methoxy-phenyl)-thiazol-2-ylamino]-ethylamino}-acetyl)-pyrrolidine-2-carbonitrile,
(2S)-1-({2-[4-(3-Phenyl-isoxazol-5-yl)-thiazol-2-ylamino]-ethylamino}-acetyl)-pyrrolidine-2-carbonitrile,
(2S)-1-{[2-(5-Methyl-2-phenyl-thiazol-4-yl)-ethylamino]-acetyl}-pyrrolidine-2-carbonitrile,
(2S)-1-({2-[2-(3-Methyl-phenyl)-5-methyl-oxazol-4-yl]-ethylamino}-acetyl)-pyrrolidine-2-carbonitrile,
(2S)-1-({2-[2-(3,3-Dimethoxy-phenyl)-3-methyl-oxazol-4-yl]-ethylamino}-acetyl)-pyrrolidine-2-carbonitrile,
(2S)-1-({2-[2-(4-Fluoro-3-methyl-phenyl)-5-methyl-oxazol-4-yl]-ethylamino}-acetyl)-pyrrolidine-2-carbonitrile,
(2S)-1-({2-[2-(3-Methyl-phenyl)-5-methyl-thiazol-4-yl]-ethylamino}-acetyl)-pyrrolidine-2-carbonitrile,
(2S)-1-({2-[2-(2-Ethyl-pyridin-4-yl)-5-methyl-thiazol-4-yl]-ethylamino}-acetyl)-pyrrolidine-2-carbonitrile,
(2S)-1-({2-[5-Methyl-2-(5-trifluoromethyl-pyridin-2-yl)-thiazol-4-yl]-ethylamino}-acetyl)-pyrrolidine-2-carbonitrile,
(2S)-1-({2-[5-Methyl-2-(6-methyl-pyridin-3-yl)-thiazol-4-yl]-ethylamino}-acetyl)-pyrrolidine-2-carbonitrile,
(2S)-1-{[1,1-Dimethyl-2-(5-methyl-2-phenyl-oxazol-4-yl)-ethylamino]-acetyl}-pyrrolidine-2-carbonitrile,
(2S)-1-({1,1-Dimethyl-2-[2-(3-methyl-phenyl)-5-methyl-oxazol-4-yl]-ethylamino}-acetyl)-pyrrolidine-2-carbonitrile,
(2S)-1-{[1-(5-Methyl-2-phenyl-oxazol-4-ylmethyl)-cyclopentylamino]-acetyl}-pyrrolidine-2-carbonitrile,
(2S)-1-{[1-(5-Methyl-2-phenyl-oxazol-4-ylmethyl)-cyclobutylamino]-acetyl}-pyrrolidine-2-carbonitrile,
(2S)-1-{[1-(5-Methyl-2-phenyl-oxazol-4-ylmethyl)-cyclopropylamino]-acetyl}-pyrrolidine-2-carbonitrile,
(2S)-1-{[1,1-Dimethyl-2-(5-methyl-2-phenyl-thiazol-4-yl)-ethylamino]-acetyl}-pyrrolidine-2-carbonitrile,
(2S)-1-{[1-(5-Methyl-2-phenyl-thiazol-4-ylmethyl)-cyclopentylamino]-acetyl}-pyrrolidine-2-carbonitrile,
(2S)-1-{[1-(5-Methyl-2-phenyl-thiazol-4-ylmethyl)-cyclobutylamino]-acetyl}-pyrrolidine-2-carbonitrile,
(2S)-1-({2-[2-(4-Fluoro-3-methyl-phenyl)-5-methyl-oxazol-4-yl]-1,1-dimethyl-ethylamino}-acetyl)-pyrrolidine-2-carbonitrile,
(2S)-1-({2-[2-(3-Chloro-phenyl)-5-methyl-oxazol-4-yl]-1,1-dimethyl-ethylamino}-acetyl)-pyrrolidine-2-carbonitrile,
(2S)-1-({2-[2-(2-Chloro-phenyl)-5-methyl-oxazol-4-yl]-1,1-dimethyl-ethylamino}-acetyl)-pyrrolidine-2-carbonitrile,
(2S)-1-({1-[2-(4-Fluoro-3-methyl-phenyl)-5-methyl-oxazol-4-ylmethyl]-cyclopropylamino}-acetyl)-pyrrolidine-2-carbonitrile,
(2S)-1-({1-[2-(3-Chloro-phenyl)-5-methyl-oxazol-4-ylmethyl]-cyclopropylamino}-acetyl)-pyrrolidine-2-carbonitrile,
(2S)-1-({1-[2-(2-Chloro-phenyl)-5-methyl-oxazol-4-ylmethyl]-cyclopropylamino}-acetyl)-pyrrolidine-2-carbonitrile,
(2S)-1-{[1,1-Dimethyl-2-(2-phenyl-oxazol-4-yl)-ethylamino]-acetyl}-pyrrolidine-2-carbonitrile,
(2S)-1-{[1,1-Dimethyl-2-(2-phenyl-thiazol-4-yl)-ethylamino]-acetyl}-pyrrolidine-2-carbonitrile,
(2S)-1-{[1,1-Dimethyl-2-(2-morpholin-4-yl-thiazol-4-yl)-ethylamino]-acetyl}-pyrrolidine-2-carbonitrile,
(2S)-1-{[1,1-Dimethyl-2-(2-piperidin-1-yl-thiazol-4-yl)-ethylamino]-acetyl}-pyrrolidine-2-carbonitrile,
(2S)-1-{[1,1-Dimethyl-3-(5-methyl-3-phenyl-pyrazol-1-yl)-propylamino]-acetyl}-pyrrolidine-2-carbonitrile, methanesulfonic acid salt,
(2S)-1-{[3-(5-Methyl-3-phenyl-pyrazol-1-yl)-propylamino]-acetyl}-pyrrolidine-2-carbonitrile, methanesulfonic acid salt,
(2S)-1-({1,1-Dimethyl-3-[5-methyl-3-(3-trifluoromethyl-phenyl)-pyrazol-1-yl]-propylamino}-acetyl)-pyrrolidine-2-carbonitrile, methanesulfonic acid salt,
(2S)-1-({1,1-Dimethyl-3-[5-methyl-3-(3-trifluoromethoxy-phenyl)-pyrazol-1-yl]-propylamino}-acetyl)-pyrrolidine-2-carbonitrile, methanesulfonic acid salt,
(2S)-1-{[3-(5-Ethyl-3-phenyl-pyrazol-1-yl)-1,1-dimethyl-propylamino]-acetyl}-pyrrolidine-2-carbonitrile, methanesulfonic acid salt,
(2S)-1-{[1,1-Dimethyl-3-(5-methyl-3-pyridin-3-yl-pyrazol-1-yl)-propylamino]-acetyl}-pyrrolidine-2-carbonitrile, methanesulfonic acid salt,
(2S)-1-{[1,1-Dimethyl-3-(3-methyl-5-pyridin-3-yl-pyrazol-1-yl)-propylamino]-acetyl}-pyrrolidine-2-carbonitrile, methanesulfonic acid salt,
(2S)-1-({3-[3-(3-Chloro-phenyl)-5-methyl-pyrazol-1-yl]-1,1-dimethyl-propylamino}-acetyl)-pyrrolidine-2-carbonitrile, methanesulfonic acid salt,
(2S)-1-({3-[3-(3,4-Dichloro-phenyl)-5-methyl-pyrazol-1-yl]-1,1-dimethyl-propylamino}-acetyl)-pyrrolidine-2-carbonitrile, methanesulfonic acid salt,
(2S)-1-{[1,1-Dimethyl-3-(3-phenyl-5-trifluoromethyl-pyrazol-1-yl)-propylamino]-acetyl}-pyrrolidine-2-carbonitrile, methanesulfonic acid salt,
(2S)-1-{[3-(5-Isopropyl-3-phenyl-pyrazol-1-yl)-1,1-dimethyl-propylamino]-acetyl}-pyrrolidine-2-carbonitrile, methanesulfonic acid salt,
(2S)-1-{[1,1-Dimethyl-3-(5-methyl-3-thiophen-2-yl-pyrazol-1-yl)-propylamino]-acetyl}-pyrrolidine-2-carbonitrile, methanesulfonic acid salt,
(2S)-1-{[1,1-Dimethyl-3-(5-methyl-3-pyridin-4-yl-pyrazol-1-yl)-propylamino]-acetyl}-pyrrolidine-2-carbonitrile, methanesulfonic acid salt,
(2S)-1-({1,1-Dimethyl-3-[5-methyl-3-(6-methyl-pyridin-3-yl)-pyrazol-1-yl]-propylamino}-acetyl)-pyrrolidine-2-carbonitrile methanesulfonic acid salt,
(2S)-1-{[3-(5-Cyclopropyl-3-phenyl-pyrazol-1-yl)-1,1-dimethyl-propylamino]-acetyl}-pyrrolidine-2-carbonitrile, methanesulfonic acid salt,
(2S)-1-{[1,1-Dimethyl-3-(5-methyl-3-pyrazin-2-yl-pyrazol-1-yl)-propylamino]-acetyl}-pyrrolidine-2-carbonitrile, methanesulfonic acid salt,
(2S)-1-({3-[3-(5-Chloro-pyridin-3-yl)-5-methyl-pyrazol-1-yl]-1,1-dimethyl-propylamino}-acetyl)-pyrrolidine-2-carbonitrile, methanesulfonic acid salt,
(2S)-1-{[1,1-Dimethyl-3-(5-methyl-3-pyridin-2-yl-pyrazol-1-yl)-propylamino]-acetyl}-pyrrolidine-2-carbonitrile, methanesulfonic acid salt,
(2S)-1-{[1,1-Dimethyl-3-(3-pyridin-3-yl-5-trifluoromethyl-pyrazol-1-yl)-propylamino]-acetyl}-pyrrolidine-2-carbonitrile, methanesulfonic acid salt,
(2S)-1-{[1,1-Dimethyl-3-(3-pyridin-3-yl-pyrazol-1-yl)-propylamino]-acetyl}-pyrrolidine-2-carbonitrile, methanesulfonic acid salt,
(2S)-1-{[1,1-Dimethyl-3-(5-methyl-3-pyridin-3-yl-[1,2,4]triazol-1-yl)-propylamino]-acetyl}-pyrrolidine-2-carbonitrile, methanesulfonic acid salt,
(2S)-1-{[1,1-Dimethyl-3-(3-pyridin-3-yl-5-trifluoromethyl-[1,2,4]triazol-1-yl)-propylamino]-acetyl}-pyrrolidine-2-carbonitrile, methanesulfonic acid salt,
(2S)-1-{[1,1-Dimethyl-3-(5-methyl-3-pyrazin-2-yl-[1,2,4]triazol-1-yl)-propylamino]-acetyl}-pyrrolidine-2-carbonitrile,
(2S)-1-{[1,1-Dimethyl-3-(2-methyl-benzoimidazol-1-yl)-propylamino]-acetyl}-pyrrolidine-2-carbonitrile,
(2S)-1-{[1,1-Dimethyl-3-(2-methyl-4-pyridin-3-yl-imidazol-1-yl)-propylamino]-acetyl}-pyrrolidine-2-carbonitrile,
(2S)-1-{[1,1-Dimethyl-3-(4-phenyl-imidazol-1-yl)-propylamino]-acetyl}-pyrrolidine-2-carbonitrile, methanesulfonic acid salt,
(2S)-1-{[1,1-Dimethyl-3-(4-pyridin-2-yl-imidazol-1-yl)-propylamino]-acetyl}-pyrrolidine-2-carbonitrile, methanesulfonic acid salt,
(2S)-1-{[1,1-Dimethyl-3-(4-pyridin-3-yl-imidazol-1-yl)-propylamino]-acetyl}-pyrrolidine-2-carbonitrile, methanesulfonic acid salt,
(2S)-1-[(6R/S)-(2-Methoxy-5,6,7,8-tetrahydro-quinolin-6-ylamino)-acetyl]-pyrrolidine-2-carbonitrile, methanesulfonic acid salt,
(2S)-1-{[1,1-Dimethyl-3-(5-cyano-2-methyl-indol-1-yl)-propylamino]-acetyl}-pyrrolidine-2-carbonitrile,
(2S)-1-{[(1S)-1-Methyl-2-(3-phenyl-pyrazol-1-yl)-ethylamino]-acetyl}-pyrrolidine-2-carbonitrile,
(2S)-1-({(1S)-2-[3-(4-Methoxy-phenyl)-pyrazol-1-yl]-1-methyl-ethylamino}-acetyl)-pyrrolidine-2-carbonitrile,
(2S)-1-({(1S)-2-[3-(4-Methoxy-phenyl)-[1,2,4]triazol-1-yl]-1-methyl-ethylamino}-acetyl)-pyrrolidine-2-carbonitrile,
(2S)-1-{[(1S)-1-Methyl-2-(5-methyl-3-phenyl-[1,2,4]triazol-1-yl)-ethylamino]-acetyl}-pyrrolidine-2-carbonitrile,
(2S)-1-{[(1S)-1-Methyl-2-(5-methyl-3-phenyl-pyrazol-1-yl)-ethylamino]-acetyl}-pyrrolidine-2-carbonitrile,
(2S)-1-{[1,1-Dimethyl-2-(5-phenyl-pyridin-2-ylamino)-ethylamino]-acetyl}-pyrrolidine-2-carbonitrile, hydrochloride salt,
(2S)-1-({2-[5-(3-Methoxy-phenyl)-pyridin-2-ylamino]-1,1-dimethyl-ethylamino}-acetyl)-pyrrolidine-2-carbonitrile, hydrochloride salt,
(2S)-1-({2-[5-(4-Cyano-phenyl)-pyridin-2-ylamino]-1,1-dimethyl-ethylamino}-acetyl)-pyrrolidine-2-carbonitrile, hydrochloride salt,
(2S)-1-({2-[5-(2-Methoxy-phenyl)-pyridin-2-ylamino]-1,1-dimethyl-ethylamino}-acetyl)-pyrrolidine-2-carbonitrile, hydrochloride salt,
(2S)-1-({2-[5-(3-Cyano-phenyl)-pyridin-2-ylamino]-1,1-dimethyl-ethylamino}-acetyl)-pyrrolidine-2-carbonitrile, hydrochloride salt,
(2S)-1-({2-[5-(3-Cyano-phenyl)-pyridin-2-ylamino]-ethylamino}-acetyl)-pyrrolidine-2-carbonitrile, hydrochloride salt,
(2S)-1-({1,1-Dimethyl-2-[5-(3-trifluoromethyl-phenyl)-pyridin-2-ylamino]-ethylamino}-acetyl)-pyrrolidine-2-carbonitrile, methansolfonic acid salt,
(2S)-1-({1,1-Dimethyl-2-[5-(4-trifluoromethyl-phenyl)-pyridin-2-ylamino]-ethylamino}-acetyl)-pyrrolidine-2-carbonitrile, methansolfonic acid salt,
(2S)-1-({1,1-Dimethyl-2-[5-(2-trifluoromethyl-phenyl)-pyridin-2-ylamino]-ethylamino}-acetyl)-pyrrolidine-2-carbonitrile, methansolfonic acid salt,
(2S)-1-({2-[5-(3,5-Bis-trifluoromethyl-phenyl)-pyridin-2-ylamino]-1,1-dimethyl-ethylamino}-acetyl)-pyrrolidine-2-carbonitrile, methansolfonic acid salt,
(2S)-1-{[2-([3,3′]Bipyridinyl-6-ylamino)-1,1-dimethyl-ethylamino]-acetyl}-pyrrolidine-2-carbonitrile, methansolfonic acid salt,
(2S)-1-({2-[5-(2,4-Dimethoxy-phenyl)-pyridin-2-ylamino]-1,1-dimethyl-ethylamino}-acetyl)-pyrrolidine-2-carbonitrile, methansolfonic acid salt,
(2S)-1-({2-[6-(4-Methoxy-phenyl)-pyridin-2-ylamino]-ethylamino}-acetyl)-pyrrolidine-2-carbonitrile, hydrochloride salt,
(2S)-1-({2-[6-(4-Cyano-phenyl)-pyridin-2-ylamino]-ethylamino}-acetyl)-pyrrolidine-2-carbonitrile, hydrochloride salt,
(2S)-1-({2-[6-(3-Methoxy-phenyl)-pyridin-2-ylamino]-ethylamino}-acetyl)-pyrrolidine-2-carbonitrile, hydrochloride salt,
(2S)-1-({2-[6-(4-Cyano-phenyl)-pyridin-2-ylamino]-1,1-dimethyl-ethylamino}-acetyl)-pyrrolidine-2-carbonitrile, hydrochloride salt,
(2S)-1-{[1,1-Dimethyl-2-(6-phenyl-pyridin-2-ylamino)-ethylamino]-acetyl}-pyrrolidine-2-carbonitrile, hydrochloride salt,
(2S)-1-({2-[6-(3-Cyano-phenyl)-pyridin-2-ylamino]-ethylamino}-acetyl)-pyrrolidine-2-carbonitrile, hydrochloride salt,
(2S)-1-({2-[6-(3-Methoxy-phenyl)-pyridin-2-ylamino]-1,1-dimethyl-ethylamino}-acetyl)-pyrrolidine-2-carbonitrile, methansolfonic acid salt,
(2S)-1-({2-[6-(4-Methoxy-phenyl)-pyridin-2-ylamino]-1,1-dimethyl-ethylamino}-acetyl)-pyrrolidine-2-carbonitrile, methansolfonic acid salt,
(2S)-1-({2-[6-(2-Methoxy-phenyl)-pyridin-2-ylamino]-1,1-dimethyl-ethylamino}-acetyl)-pyrrolidine-2-carbonitrile, methansolfonic acid salt,
(2S)-1-({2-[6-(2-Methoxy-phenyl)-pyridin-2-ylamino]-ethylamino}-acetyl)-pyrrolidine-2-carbonitrile, methansolfonic acid salt,
(2S)-1-({2-[6-(3-Cyano-phenyl)-pyridin-2-ylamino]-1,1-dimethyl-ethylamino}-acetyl)-pyrrolidine-2-carbonitrile, methansolfonic acid salt,
(2S)-1-({2-[6-(3,3-Bis-trifluoromethyl-phenyl)-pyridin-2-ylamino]-1,1-dimethyl-ethylamino}-acetyl)-pyrrolidine-2-carbonitrile, methansolfonic acid salt,
(2S)-1-({1,1-Dimethyl-2-[6-(4-trifluoromethyl-phenyl)-pyridin-2-ylamino]-ethylamino}-acetyl)-pyrrolidine-2-carbonitrile, methansolfonic acid salt,
(2S)-1-({1,1-Dimethyl-2-[6-(2-trifluoromethyl-phenyl)-pyridin-2-ylamino]-ethylamino}-acetyl)-pyrrolidine-2-carbonitrile, methansolfonic acid salt,
(2S)-1-({1,1-Dimethyl-2-[6-(3-trifluoromethyl-phenyl)-pyridin-2-ylamino]-ethylamino}-acetyl)-pyrrolidine-2-carbonitrile, methansolfonic acid salt,
(2S)-1-{[2-([2,3′]Bipyridinyl-6-ylamino)-1,1-dimethyl-ethylamino]-acetyl}-pyrrolidine-2-carbonitrile, methansolfonic acid salt,
(2S)-1-({2-[6-(2,4-Dimethoxy-phenyl)-pyridin-2-ylamino]-1,1-dimethyl-ethylamino}-acetyl)-pyrrolidine-2-carbonitrile, methansolfonic acid salt,
(2S)-1-{[1,1-Dimethyl-2-(6-m-tolyl-pyridin-2-ylamino)-ethylamino]-acetyl}-pyrrolidine-2-carbonitrile, methanesulfonic acid salt,
(2S)-1-{[1,1-Dimethyl-2-(5-phenyl-pyrimidin-2-ylamino)-ethylamino]-acetyl}-pyrrolidine-2-carbonitrile,
(2S)-1-({2-[5-(3-Methoxy-phenyl)-pyrimidin-2-ylamino]-1,1-dimethyl-ethylamino}-acetyl)-pyrrolidine-2-carbonitrile,
(2S)-1-({2-[5-(3-Cyano-phenyl)-pyrimidin-2-ylamino]-1,1-dimethyl-ethylamino}-acetyl)-pyrrolidine-2-carbonitrile,
(2S)-1-({2-[5-(4-Cyano-phenyl)-pyrimidin-2-ylamino]-1,1-dimethyl-ethylamino}-acetyl)-pyrrolidine-2-carbonitrile,
(2S)-1-({2-[4-(2,4-Dimethoxy-phenyl)-thiazol-2-ylamino]-ethylamino}-acetyl)-pyrrolidine-2-carbonitrile,
(2S)-1-({2-[4-(2-Methoxy-phenyl)-thiazol-2-ylamino]-ethylamino}-acetyl)-pyrrolidine-2-carbonitrile,
(2S)-1-{[2-(4-Phenyl-thiazol-2-ylamino)-ethylamino]-acetyl}-pyrrolidine-2-carbonitrile,
(2S)-1-({2-[4-(3-Methoxy-phenyl)-thiazol-2-ylamino]-ethylamino}-acetyl)-pyrrolidine-2-carbonitrile,
(2S)-1-{[2-(8H-Indeno[1,2-d]thiazol-2-ylamino)-ethylamino]-acetyl}-pyrrolidine-2-carbonitrile, hydrochloride salt,
(2S)-1-{[2-(5-Methyl-4-phenyl-thiazol-2-ylamino)-ethylamino]-acetyl}-pyrrolidine-2-carbonitrile, hydrochloride salt,
(2S)-1-{[2-(4,5-Diphenyl-thiazol-2-ylamino)-ethylamino]-acetyl}-pyrrolidine-2-carbonitrile, hydrochloride salt,
(2S)-1-{[2-(4-Benzoyl-thiazol-2-ylamino)-ethylamino]-acetyl}-pyrrolidine-2-carbonitrile,
(2S)-1-({2-[4-(4-Fluoro-phenyl)-thiazol-2-ylamino]-ethylamino}-acetyl)-pyrrolidine-2-carbonitrile,
(2S)-1-({2-[4-(4-Trifluoromethyl-phenyl)-thiazol-2-ylamino]-ethylamino}-acetyl)-pyrrolidine-2-carbonitrile,
(2S)-1-{[2-(4-Pyridin-2-yl-thiazol-2-ylamino)-ethylamino]-acetyl}-pyrrolidine-2-carbonitrile,
(2S)-1-{[2-(4-Pyridin-4-yl-thiazol-2-ylamino)-ethylamino]-acetyl}-pyrrolidine-2-carbonitrile,
(2S)-1-({2-[5-Methyl-4-(4-trifluoromethyl-phenyl)-thiazol-2-ylamino]-ethylamino}-acetyl)-pyrrolidine-2-carbonitrile,
(2S)-1-({2-[4-(4-Cyano-phenyl)-5-methyl-thiazol-2-ylamino]-ethylamino}-acetyl)-pyrrolidine-2-carbonitrile,
(2S)-1-{[2-(4-Pyridin-3-yl-thiazol-2-ylamino)-ethylamino]-acetyl}-pyrrolidine-2-carbonitrile,
(2S)-1-({2-[4-(4-Cyano-phenyl)-thiazol-2-ylamino]-1,1-dimethyl-ethylamino}-acetyl)-pyrrolidine-2-carbonitrile, methanesulfonic acid salt,
(2S)-1-{[2-(4,5,6,7-Tetrahydro-benzothiazol-2-ylamino)-ethylamino]-acetyl}-pyrrolidine-2-carbonitrile,
(2S)-1-{[1,1-dimethyl-2-(6-ethoxycarbonyl-4,5,6,7-tetrahydro-thiazolo[5,4-c]pyridine-2-ylamino)-ethylamino]-acetyl}-pyrrolidine-2-carbonitrile, methanesulfonic acid salt,
(2S)-1-{[1,1-dimethyl-2-(6-acetyl-4,5,6,7-tetrahydro-thiazolo[5,4-c]pyridine-2-ylamino)-ethylamino]-acetyl}-pyrrolidine-2-carbonitrile, methanesulfonic acid salt,
(2S)-1-{[2-(Benzothiazol-2-ylamino)-1,1-dimethyl-ethylamino]-acetyl}-pyrrolidine-2-carbonitrile,
(2S)-1-{[2-(Benzothiazol-2-ylamino)-ethylamino]-acetyl}-pyrrolidine-2-carbonitrile,
(2S)-1-{[2-(Benzooxazol-2-ylamino)-ethylamino]-acetyl}-pyrrolidine-2-carbonitrile,
(2S)-1-{[2-(Benzooxazol-2-ylamino)-1,1-dimethyl-ethylamino]-acetyl}-pyrrolidine-2-carbonitrile,
(2S)-1-{[1,1-Dimethyl-2-(1-methyl-1H-benzoimidazol-2-ylamino)-ethylamino]-acetyl}-pyrrolidine-2-carbonitrile,
(2S)-1-{[1,1-Dimethyl-2-(5-phenyl-[1,3,4]oxadiazol-2-ylamino)-ethylamino]-acetyl}-pyrrolidine-2-carbonitrile, methanesulfonic acid salt,
(2S)-1-{[1,1-Dimethyl-2-(3-pyridin-3-yl-[1,2,4]oxadiazol-5-ylamino)-ethylamino]-acetyl}-pyrrolidine-2-carbonitrile, methanesulfonic acid salt,
(2S)-1-{[1,1-Dimethyl-2-(3-phenyl-[1,2,4]oxadiazol-5-ylamino)-ethylamino]-acetyl}-pyrrolidine-2-carbonitrile, methanesulfonic acid salt,
(2S)-1-{[1,1-Dimethyl-2-(3-pyridin-2-yl-[1,2,4]oxadiazol-5-ylamino)-ethylamino]-acetyl}-pyrrolidine-2-carbonitrile, methanesulfonic acid salt,
(2S)-1-{[1,1-Dimethyl-2-(3-pyridin-4-yl-[1,2,4]oxadiazol-5-ylamino)-ethylamino]-acetyl}-pyrrolidine-2-carbonitrile, methanesulfonic acid salt,
(2S)-1-({1,1-Dimethyl-2-[3-(6-methyl-pyridin-3-yl)-[1,2,4]oxadiazol-5-ylamino]-ethylamino}-acetyl)-pyrrolidine-2-carbonitrile, methanesulfonic acid salt,
(2S)-1-({2-[3-(2-Chloro-pyridin-4-yl)-[1,2,4]oxadiazol-5-ylamino]-1,1-dimethyl-ethylamino}-acetyl)-pyrrolidine-2-carbonitrile, methanesulfonic acid salt,
(2S)-1-({2-[3-(3,5-Dichloro-phenyl)-[1,2,4]oxadiazol-5-ylamino]-1,1-dimethyl-ethylamino}-acetyl)-pyrrolidine-2-carbonitrile, methanesulfonic acid salt,
(2S)-1-{[3-(2-Phenyl-1H-imidazol-4-yl)-propylamino]-acetyl}-pyrrolidine-2-carbonitrile,
(2S)-1-{[(5-Methyl-2-phenyl-1H-imidazol-4-ylmethyl)-amino]-acetyl}-pyrrolidine-2-carbonitrile,
(2S)-1-{[2-(5-Methyl-2-phenyl-1H-imidazol-4-yl)-ethylamino]-acetyl}-pyrrolidine-2-carbonitrile,
(2S)-1-{[2-(5-Methyl-2-pyridin-4-yl-1H-imidazol-4-yl)-ethylamino]-acetyl}-pyrrolidine-2-carbonitrile,
(2S)-1-{[2-(5-Methyl-2-pyridin-3-yl-1H-imidazol-4-yl)-ethylamino]-acetyl}-pyrrolidine-2-carbonitrile,
(2S)-1-{[2-(5-Methyl-2-pyridin-2-yl-1H-imidazol-4-yl)-ethylamino]-acetyl}-pyrrolidine-2-carbonitrile,
(2S)-1-{[2-(2-Phenyl-1H-imidazol-4-yl)-ethylamino]-acetyl}-pyrrolidine-2-carbonitrile,
(2S)-1-({2-[2-(3-Fluoro-4-methyl-phenyl)-5-methyl-1H-imidazol-4-yl]-1,1-dimethyl-ethylamino}-acetyl)-pyrrolidine-2-carbonitrile,
(2S)-1-({1,1-Dimethyl-2-[5-methyl-2-(4-trifluoromethyl-phenyl)-1H-imidazol-4-yl]-ethylamino}-acetyl)-pyrrolidine-2-carbonitrile,
(2S)-1-{[1,1-Dimethyl-2-(5-methyl-2-m-tolyl-1H-imidazol-4-yl)-ethylamino]-acetyl}-pyrrolidine-2-carbonitrile,
(2S)-1-({1,1-Dimethyl-2-[5-methyl-2-(3-chlorophenyl)-1H-imidazol-4-yl]-ethylamino}-acetyl)-pyrrolidine-2-carbonitrile,
(2S)-1-({2-[2-(3,5-Bis-trifluoromethyl-phenyl)-5-methyl-1H-imidazol-4-yl]-1,1-dimethyl-ethylamino}-acetyl)-pyrrolidine-2-carbonitrile,
(2S)-1-({2-[2-(3,5-Dichloro-phenyl)-5-methyl-1H-imidazol-4-yl]-1,1-dimethyl-ethylamino}-acetyl)-pyrrolidine-2-carbonitrile,
(2S)-1-{[1,1-Dimethyl-2-(2-phenyl-1H-imidazol-4-yl)-ethylamino]-acetyl}-pyrrolidine-2-carbonitrile,
(2S)-1-{[1,1-Dimethyl-2-(1-methyl-2-phenyl-1H-imidazol-4-yl)-ethylamino]-acetyl}-pyrrolidine-2-carbonitrile,
(2S)-1-{[2-(1,5-Dimethyl-2-phenyl-1H-imidazol-4-yl)-1,1-dimethyl-ethylamino]-acetyl}-pyrrolidine-2-carbonitrile,
(2S)-1-({2-[2-(3-Fluoro-phenyl)-5-methyl-1H-imidazol-4-yl]-1,1-dimethyl-ethylamino}-acetyl)-pyrrolidine-2-carbonitrile,
(2S)-1-({2-[2-(3-Methoxy-phenyl)-5-methyl-1H-imidazol-4-yl]-1,1-dimethyl-ethylamino}-acetyl)-pyrrolidine-2-carbonitrile,
(2S)-1-({2-[2-(3-Ethoxy-phenyl)-5-methyl-1H-imidazol-4-yl]-1,1-dimethyl-ethylamino}-acetyl)-pyrrolidine-2-carbonitrile,
(2S)-1-({2-[2-(3,5-Difluoro-phenyl)-5-methyl-1H-imidazol-4-yl]-1,1-dimethyl-ethylamino}-acetyl)-pyrrolidine-2-carbonitrile,
(2S)-1-({2-[2-(3,5-Dimethoxy-phenyl)-5-methyl-1H-imidazol-4-yl]-1,1-dimethyl-ethylamino}-acetyl)-pyrrolidine-2-carbonitrile,
(2S)-1-({1,1-Dimethyl-2-[5-methyl-2-(3-trifluoromethyl-phenyl)-1H-imidazol-4-yl]-ethylamino}-acetyl)-pyrrolidine-2-carbonitrile,
(2S)-1-{[1,1-Dimethyl-2-(5-methyl-2-pyridin-2-yl-1H-imidazol-4-yl)-ethylamino]-acetyl}-pyrrolidine-2-carbonitrile,
(2S)-1-{[1,1-Dimethyl-2-(5-methyl-2-pyridin-3-yl-1H-imidazol-4-yl)-ethylamino]-acetyl}-pyrrolidine-2-carbonitrile,
(2S)-1-{[1,1-Dimethyl-2-(5-methyl-2-pyridin-4-yl-1H-imidazol-4-yl)-ethylamino]-acetyl}-pyrrolidine-2-carbonitrile,
(2S)-1-({1,1-Dimethyl-2-[5-methyl-2-(3-trifluoromethoxy-phenyl)-1H-imidazol-4-yl]-ethylamino}-acetyl)-pyrrolidine-2-carbonitrile,
(2S)-1-{[1,1-Dimethyl-2-(5-methyl-2-phenyl-1H-imidazol-4-yl)-ethylamino]-acetyl}-pyrrolidine-2-carbonitrile,
(2S)-1-({2-[2-(4-Chloro-phenyl)-5-methyl-1H-imidazol-4-yl]-1,1-dimethyl-ethylamino}-acetyl)-pyrrolidine-2-carbonitrile,
(2S)-1-{[1,1-Dimethyl-2-(5-methyl-2-p-tolyl-1H-imidazol-4-yl)-ethylamino]-acetyl}-pyrrolidine-2-carbonitrile,
(2S)-1-({2-[2-(3-Chloro-4-methyl-phenyl)-5-methyl-1H-imidazol-4-yl]-1,1-dimethyl-ethylamino}-acetyl)-pyrrolidine-2-carbonitril, and
(2S)-1-({1,1-Dimethyl-2-[2-(3-acetamidophenyl)-5-methyl-1H-imidazol-4-yl]-ethylamino}-acetyl)-pyrrolidine-2-carbonitrile,
   and pharmaceutically acceptable salts thereof.

Preferably, the DPP-IV inhibitor according to formula (I) is selected from the group consisting of
(2S)-1-({2-[5-(5-Methyl-[1,3,4]oxadiazol-2-yl)-pyridin-2-ylamino]-ethylamino}-acetyl)-pyrrolidine-2-carbonitrile,
(2S)-1-{[(1S)-2-(5-cyano-2-methyl-indol-1-yl)-1-methyl-ethylamino]-acetyl}-pyrrolidine-2-carbonitrile,
(2S)-1-{[2-(5-Methyl-2-phenyl-oxazol-4-yl)-ethylamino]-acetyl}-pyrrolidine-2-carbonitrile,
(2S)-1-[((2R/S)-6-Methoxy-1,2,3,4-tetrahydro-naphthalen-2-ylamino)-acetyl]-pyrrolidine-2-carbonitrile,
(2S)-1-({2-[2-(4-Fluoro-phenyl)-5-methyl-oxazol-4-yl]-ethylamino}-acetyl)-pyrrolidine-2-carbonitrile,
(2S)-1-({2-[5-(4-Methoxy-phenyl)-pyridin-2-ylamino]-1,1-dimethyl-ethylamino}-acetyl)-pyrrolidine-2-carbonitrile,
(2S)-1-({2-[4-(4-Cyano-phenyl)-thiazol-2-ylamino]-ethylamino}-acetyl)-pyrrolidine-2-carbonitrile,
(2S)-1-({2-[5-(3-Methoxy-phenyl)-pyridin-2-ylamino]-ethylamino}-acetyl)-pyrrolidine-2-carbonitrile,
(2S)-1-{[(1S)-2-(5-Methoxy-2-methyl-indol-1-yl)-1-methyl-ethylamino]-acetyl}-pyrrolidine-2-carbonitrile,
(2S)-1-({2-[5-(4-Cyano-phenyl)-pyridin-2-ylamino]-ethylamino}-acetyl)-pyrrolidine-2-carbonitrile,
(2S)-1-({2-[5-Phenyl-pyridin-2-ylamino]-ethylamino}-acetyl)-pyrrolidine-2-carbonitrile,
(2S)-1-({2-[4-(3-Phenyl-isoxazol-5-yl)-thiazol-2-ylamino]-ethylamino}-acetyl)-pyrrolidine-2-carbonitrile,
(2S)-1-{[(1S)-1-Methyl-2-(2-methyl-indol-1-yl)-ethylamino]-acetyl}-pyrrolidine-2-carbonitrile,
(2S)-1-({2-[5-(4-Methoxy-phenyl)-pyridin-2-ylamino]-ethylamino}-acetyl)-pyrrolidine-2-carbonitrile,
(2S)-1-({2-[2-(4-Benzyloxy-phenyl)-5-methyl-oxazol-4-yl]-ethylamino}-acetyl)-pyrrolidine-2-carbonitrile,
(2S)-1-{[(1S)-2-(2,3-Dimethyl-indol-1-yl)-1-methyl-ethylamino]-acetyl}-pyrrolidine-2-carbonitrile,
(2S)-1-({2-[5-(2-Methoxy-phenyl)-pyridin-2-ylamino]-ethylamino}-acetyl)-pyrrolidine-2-carbonitrile,
(2S)-1-{[(1S)-2-(5-cyano-indol-1-yl)-1-methyl-ethylamino]-acetyl}-pyrrolidine-2-carbonitrile,
(2S)-1-{[1,1-Dimethyl-2-(5-methyl-2-phenyl-oxazol-4-yl)-ethylamino]-acetyl}-pyrrolidine-2-carbonitrile,
(2S)-1-{[1,1-Dimethyl-3-(5-methyl-3-pyridin-3-yl-pyrazol-1-yl)-propylamino]-acetyl}-pyrrolidine-2-carbonitrile,
(2S)-1-{[1,1-Dimethyl-3-(5-methyl-3-pyrazin-2-yl-pyrazol-1-yl)-propylamino]-acetyl}-pyrrolidine-2-carbonitrile,
(2S)-1-{[1,1-Dimethyl-3-(3-pyridin-3-yl-pyrazol-1-yl)-propylamino]-acetyl}-pyrrolidine-2-carbonitrile,
(2S)-1-{[1,1-Dimethyl-3-(5-methyl-3-pyridin-3-yl-[1,2,4]triazol-1-yl)-propylamino]-acetyl}-pyrrolidine-2-carbonitrile,
(2S)-1-{[1,1-Dimethyl-3-(2-methyl-4-pyridin-3-yl-imidazol-1-yl)-propylamino]-acetyl}-pyrrolidine-2-carbonitrile,
(2S)-1-{[1,1-Dimethyl-3-(4-pyridin-3-yl-imidazol-1-yl)-propylamino]-acetyl}-pyrrolidine-2-carbonitrile,
(2S)-1-{[1,1-dimethyl-2-(6-acetyl-4,5,6,7-tetrahydro-thiazolo[5,4-c]pyridine-2-ylamino)-ethylamino]-acetyl}-pyrrolidine-2-carbonitrile,
(2S)-1-{[2-(Benzothiazol-2-ylamino)-1,1-dimethyl-ethylamino]-acetyl}-pyrrolidine-2-carbonitrile,
(2S)-1-{[1,1-Dimethyl-2-(5-phenyl-[1,3,4]oxadiazol-2-ylamino)-ethylamino]-acetyl}-pyrrolidine-2-carbonitrile,
(2S)-1-{[1,1-Dimethyl-2-(3-pyridin-3-yl-[1,2,4]oxadiazol-5-ylamino)-ethylamino]-acetyl}-pyrrolidine-2-carbonitrile,
(2S)-1-{[1,1-Dimethyl-2-(3-pyridin-2-yl-[1,2,4]oxadiazol-5-ylamino)-ethylamino]-acetyl}-pyrrolidine-2-carbonitrile,
(2S)-1-{[1,1-Dimethyl-2-(3-pyridin-4-yl-[1,2,4]oxadiazol-5-ylamino)-ethylamino]-acetyl}-pyrrolidine-2-carbonitrile, and
(2S)-1-({1,1-Dimethyl-2-[3-(6-methyl-pyridin-3-yl)-[1,2,4]oxadiazol-5-ylamino]-ethylamino}-acetyl)-pyrrolidine-2-carbonitrile,
   and pharmaceutically acceptable salts thereof.

More preferably, the DPP-IV inhibitor of formula (I) is
(2S)-1-{[2-(5-Methyl-2-phenyl-oxazol-4-yl)-ethylamino]-acetyl}-pyrrolidine-2-carbonitrile, or
(2S)-1-{[1,1-Dimethyl-3-(4-pyridin-3-yl-imidazol-1-yl)-propylamino]-acetyl}-pyrrolidine-2-carbonitrile,
   and pharmaceutically acceptable salts thereof.

(2S)-1-{[2-(5-Methyl-2-phenyl-oxazol-4-yl)-ethylamino]-acetyl}-pyrrolidine-2-carbonitrile is preferably used in form of the mesylate salt.

The compounds of formula (I) and methods for their preparation have been disclosed and described in WO 03/037327.

In addition, in the pharmaceutical compositions according to the present invention, the DPP-IV inhibitor can preferably be a compound of formula (II)

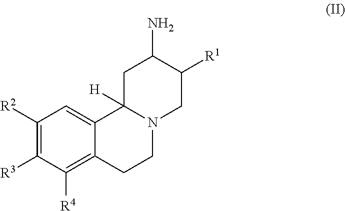

wherein

### R¹ is —C(O)—N(R⁵)R⁶ or —N(R⁵)R⁶;

R², R³ and R⁴ are each independently hydrogen, halogen, hydroxy, lower alkyl, lower alkoxy or lower alkenyl, wherein lower alkyl, lower alkoxy and lower alkenyl may optionally be substituted by lower alkoxycarbonyl, aryl or heterocyclyl;
R⁵ is hydrogen, lower alkyl, halogenated lower alkyl or cycloalkyl;
R⁶ is lower alkylsulfonyl, halogenated lower alkylsulfonyl, cycloalkylsulfonyl, lower alkylcarbonyl, halogenated lower alkylcarbonyl, cycloalkylcarbonyl; or
R⁵ and R⁶ together with the nitrogen atom to which they are attached form a 4-, 5-, 6- or 7-membered saturated or unsaturated heterocyclic ring optionally containing a further heteroatom selected from nitrogen, oxygen and sulfur, said heterocyclic ring being optionally mono-, di-, or tri-substituted, independently, with lower alkyl, halogenated lower alkyl, oxo, dioxo and/or cyano;
and pharmaceutically acceptable salts thereof.

DPP-IV inhibitors according to formula (II) preferably include those selected from the group consisting of
(RS,RS,RS)-(2-amino-9,10-dimethoxy-1,3,4,6,7,11b-hexahydro-2H-pyrido[2,1-a]isoquinolin-3-yl)-pyrrolidin-1-yl-methanone,
(RS,RS,RS)-(2-amino-9,10-dimethoxy-1,3,4,6,7,11b-hexahydro-2H-pyrido[2,1-a]isoquinolin-3-yl)-thiazolidin-3-yl-methanone,
(RS,RS,RS)-(2-amino-9,10-dimethoxy-1,3,4,6,7,11b-hexahydro-2H-pyrido[2,1-a]isoquinolin-3-yl)-azetidin-1-yl-methanone,
(SS)-1-((RS,RS,RS)-2-amino-9,10-dimethoxy-1,3,4,6,7,11b-hexahydro-2H-pyrido[2,1-a]isoquinoline-3-carbonyl)-pyrrolidine-2-carbonitrile,
1-((RS,RS,RS)-2-amino-9,10-dimethoxy-1,3,4,6,7,11b-hexahydro-2H-pyrido[2,1-a]isoquinolin-3-yl)-piperidin-2-one,
(−)-(S,S,S)-1-(2-amino-9,10-dimethoxy-1,3,4,6,7,11b-hexahydro-2H-pyrido[2,1-a]isoquinolin-3-yl)-piperidin-2-one,
(+)-(R,R,R)-1-(2-amino-9,10-dimethoxy-1,3,4,6,7,11b-hexahydro-2H-pyrido[2,1-a]isoquinolin-3-yl)-piperidin-2-one,
1-((RS,RS,RS)-2-amino-9,10-dimethoxy-1,3,4,6,7,11b-hexahydro-2H-pyrido[2,1-a]isoquinolin-3-yl)-4-methyl-piperidin-2-one,
(RS,RS,RS)-1-(2-amino-9,10-dimethoxy-1,3,4,6,7,11b-hexahydro-2H-pyrido[2,1-a]isoquinolin-3-yl)-pyrrolidin-2-one,
1-((RS,RS,RS)-2-amino-9,10-dimethoxy-1,3,4,6,7,11b-hexahydro-2H-pyrido[2,1-a]isoquinolin-3-yl)-4-methyl-pyrrolidin-2-one,
1-((RS,RS,RS)-2-amino-9,10-dimethoxy-1,3,4,6,7,11b-hexahydro-2H-pyrido[2,1-a]isoquinolin-3-yl)-4-ethyl-pyrrolidin-2-one,
(RS,RS,RS)-1-(2-amino-9,10-dimethoxy-1,3,4,6,7,11b-hexahydro-2H-pyrido[2,1-a]isoquinolin-3-yl)-5,6-dihydro-1H-pyridin-2-one,
1-((RS,RS,RS)-2-amino-9,10-dimethoxy-1,3,4,6,7,11b-hexahydro-2H-pyrido[2,1-a]isoquinolin-3-yl)-azepan-2-one,
(RS,RS,RS)-3-(1,1-dioxo-1,2-thiazolidin-2-yl)-9,10-dimethoxy-1,3,4,6,7,11b-hexahydro-2H-pyrido[2,1-a]isoquinolin-2-ylamine,
(RS,RS,RS)-3-(1,1-dioxo[1,2]thiazinan-2-yl)-9,10-dimethoxy-1,3,4,6,7,11b-hexahydro-2H-pyrido[2,1-a]isoquinolin-2-ylamine,
(S,S,S)-3-(1,1-dioxo-[1,2]thiazinan-2-yl)-9,10-dimethoxy-1,3,4,6,7,11b-hexahydro-2H-pyrido[2,1-a]isoquinolin-2-ylamine,
(SR)-1-((RS,RS,RS)-2-amino-9,10-dimethoxy-1,3,4,6,7,11b-hexahydro-2H-pyrido[2,1-a]isoquinolin-3-yl)-4-methyl-pyrrolidin-2-one,
(RS,RS,RS,RS)-1-(2-amino-9,10-dimethoxy-1,3,4,6,7,11b-hexahydro-2H-pyrido[2,1-a]isoquinolin-3-yl)-4-methyl-pyrrolidin-2-one,
(R)-1-((S,S,S)-2-amino-9,10-dimethoxy-1,3,4,6,7,11b-hexahydro-2H-pyrido[2,1-a]isoquinolin-3-yl)-4-methyl-pyrrolidin-2-one,
(S)-1-((R,R,R)-2-amino-9,10-dimethoxy-1,3,4,6,7,11b-hexahydro-2H-pyrido[2,1-a]isoquinolin-3-yl)-4-methyl-pyrrolidin-2-one,
(S,S,S,S)-1-(2-amino-9,10-dimethoxy-1,3,4,6,7,11b-hexahydro-2H-pyrido[2,1-a]isoquinolin-3-yl)-4-methyl-pyrrolidin-2-one,
(R,R,R,R)-1-(2-amino-9,10-dimethoxy-1,3,4,6,7,11b-hexahydro-2H-pyrido[2,1-a]isoquinolin-3-yl)-4-methyl-pyrrolidin-2-one,
1-((RS,RS,RS)-2-amino-9,10-dimethoxy-1,3,4,6,7,11b-hexahydro-2H-pyrido[2,1-a]isoquinolin-3-yl)-4-fluoromethyl-pyrrolidin-2-one,
1-((RS,RS,RS)-2-amino-9,10-dimethoxy-1,3,4,6,7,11b-hexahydro-2H-pyrido[2,1-a]isoquinolin-3-yl)-5-methyl-piperidin-2-one,
(RS,RS,RS)—N-(2-amino-9,10-dimethoxy-1,3,4,6,7,11b-hexahydro-2H-pyrido[2,1-a]isoquinolin-3-yl)-propionamide,
(RS,RS,RS)—N-(2-amino-9,10-dimethoxy-1,3,4,6,7,11b-hexahydro-2H-pyrido[2,1-a]isoquinolin-3-yl)-butyramide,
cyclopropanecarboxylic acid ((2RS,3RS,11bRS)-2-amino-9,10-dimethoxy-1,3,4,6,7,11b-hexahydro-2H-pyrido[2,1-a]isoquinolin-3-yl)-amide,
(SR)-1-((RS,RS,RS)-2-amino-9,10-dimethoxy-1,3,4,6,7,11b-hexahydro-2H-pyrido[2,1-a]isoquinolin-3-yl)-4-fluoromethyl-pyrrolidin-2-one,
(RS,RS,RS,RS)-1-(2-amino-9,10-dimethoxy-1,3,4,6,7,11b-hexahydro-2H-pyrido[2,1-a]isoquinolin-3-yl)-4-fluoromethyl-pyrrolidin-2-one,
(S)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one,
(R)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one,
3-((RS,RS,RS)-2-amino-9,10-dimethoxy-1,3,4,6,7,11b-hexahydro-2H-pyrido[2,1-a]isoquinolin-3-yl)-oxazolidin-2-one,
3-((2RS,3RS,11bRS)-2-amino-9,10-dimethoxy-1,3,4,6,7,11b-hexahydro-2H-pyrido[2,1-a]isoquinolin-3-yl)-[1,3]oxazinan-2-one,
1-((2RS,3RS,11bRS)-2-amino-9,10-dimethoxy-1,3,4,6,7,11b-hexahydro-2H-pyrido[2,1-a]isoquinolin-3-yl)-5-methyl-pyrrolidin-2-one,
3-((2RS,3RS,11bRS)-2-amino-9,10-dimethoxy-1,3,4,6,7,11b-hexahydro-2H-pyrido[2,1-a]isoquinolin-3-yl)-5-fluoromethyl-oxazolidin-2-one,
1-((2RS,3RS,11bRS)-2-amino-9,10-dimethoxy-1,3,4,6,7,11b-hexahydro-2H-pyrido[2,1-a]isoquinolin-3-yl)-3-methyl-pyrrolidin-2-one, and
3-((2RS,3RS,11bRS)-2-amino-9,10-dimethoxy-1,3,4,6,7,11b-hexahydro-2H-pyrido[2,1-a]isoquinolin-3-yl)-5-methyl-oxazolidin-2-one,
   and pharmaceutically acceptable salts thereof.

Preferably, the DPP-IV inhibitor of formula (II) is selected from the group consisting of
(RS,RS,RS)-(2-amino-9,10-dimethoxy-1,3,4,6,7,11b-hexahydro-2H-pyrido[2,1-a]isoquinolin-3-yl)-thiazolidin-3-yl-methanone,
(−)-(S,S,S)-1-(2-amino-9,10-dimethoxy-1,3,4,6,7,11b-hexahydro-2H-pyrido[2,1-a]isoquinolin-3-yl)-piperidin-2-one,
1-((RS,RS,RS)-2-amino-9,10-dimethoxy-1,3,4,6,7,11b-hexahydro-2H-pyrido[2,1-a]isoquinolin-3-yl)-4-methyl-pyrrolidin-2-one,
(RS,RS,RS)-1-(2-amino-9,10-dimethoxy-1,3,4,6,7,11b-hexahydro-2H-pyrido[2,1-a]isoquinolin-3-yl)-5,6-dihydro-1H-pyridin-2-one,
(S,S,S)-3-(1,1-dioxo-[1,2]thiazinan-2-yl)-9,10-dimethoxy-1,3,4,6,7,11b-hexahydro-2H-pyrido[2,1-a]isoquinolin-2-ylamine,
(R)-1-((S,S,S)-2-amino-9,10-dimethoxy-1,3,4,6,7,11b-hexahydro-2H-pyrido[2,1-a]isoquinolin-3-yl)-4-methyl-pyrrolidin-2-one,
(S,S,S,S)-1-(2-amino-9,10-dimethoxy-1,3,4,6,7,11b-hexahydro-2H-pyrido[2,1-a]isoquinolin-3-yl)-4-methyl-pyrrolidin-2-one,
1-((RS,RS,RS)-2-amino-9,10-dimethoxy-1,3,4,6,7,11b-hexahydro-2H-pyrido[2,1-a]isoquinolin-3-yl)-5-methyl-piperidin-2-one,
(S)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one,
(R)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one,
3-((2RS,3RS,11bRS)-2-amino-9,10-dimethoxy-1,3,4,6,7,11b-hexahydro-2H-pyrido[2,1-a]isoquinolin-3-yl)-5-methyl-oxazolidin-2-one,
   and pharmaceutically acceptable salts thereof.

More preferably, the DPP-IV inhibitor of formula (II) is
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one, or
(S,S,S,S)-1-(2-Amino-9,10-dimethoxy-1,3,4,6,7,11b-hexahydro-2H-pyrido[2,1-a]isoquinolin-3-yl)-4-methyl-pyrrolidin-2-one,
   and pharmaceutically acceptable salts thereof. (S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one and pharmaceutically acceptable salts thereof is preferred.

The compounds of formula (II) and methods for their preparation have been described in WO 2005/000848.

In addition, in the pharmaceutical compositions according to the present invention, the DPP-IV inhibitor can preferably be a compound of formula (IIIA) or (IIIB)

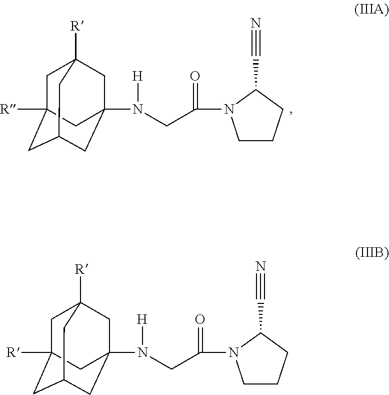

wherein R′ represents hydroxy, C₁-C₇alkoxy, C₁-C₈-alkanoyloxy, or R₅R₄N—CO—O—, where R₄ and R₅ independently are C₁-C₇alkyl or phenyl which is unsubstituted or substituted by a substitutent selected from C₁-C₇alkyl, C₁-C₇alkoxy, halogen and trifluoromethyl and where R₄ additionally is hydrogen; or R₄ and R₅ together represent C₃-C₆ alkylene; and R″ represents hydrogen; or R′ and R″ independently represent C₁-C₇ alkyl; in free form or in form of a pharmaceutically acceptable acid addition salt.

The DPP-IV inhibitors of formula (IIIA) or (IIIB) have been disclosed and described in detail in WO00/34241.

Preferably, the DPP-IV inhibitor of formula (IIIA) or (IIIB) is selected from the compounds specifically described in WO00/34241.

Preferably, the DPP-IV inhibitor of formula (IIIA) or (IIIB) is selected from the group consisting of
pyrrolidine, 1-[[(3,5-dimethyl-1-adamantyl)amino]-acetyl]-2-cyano-, (S)—;
pyrrolidine, 1-[[(3-ethyl-1-adamantyl)amino]acetyl]-2-cyano-, (S)—;
pyrrolidine, 1-[[(3-methoxy-1-adamantyl)amino]-acetyl]-2-cyano-, (S)—;
pyrrolidine, 1-[[[3-[[(t-butylamino)carbonyl]oxy]-1-adamantyl]amino]acetyl]-2-cyano-, (S)—;
pyrrolidine, 1-[[[3-[[[(4-methoxyphenyl)amino]-carbonyl]oxy]-1-adamantyl]amino]acetyl]-2-cyano-, (S)—;
pyrrolidine, 1-[[[(3-[[(phenylamino)carbonyl]oxy]-1-adamantyl]amino]acetyl]-2-cyano-, (S)—;
pyrrolidine, 1-[[(3-hydroxy-2-adamantyl)amino]-acetyl]-2-cyano-, (S)—;
pyrrolidine, 1-[[(3-acetyloxy-1-adamantyl)amino]acetyl]-2-cyano-, (S)—;
pyrrolidine, 1-[[[3-[[[(diisopropyl)amino]carbonyl]oxy]-1-adamantyl]amino]acetyl]-2-cyano-, (S)—;
pyrrolidine, 1-[[[3-[[[(cyclohexyl)amino]carbonyl]oxy]-1-adamantyl]amino]acetyl]-2-cyano-, (S)—; and
pyrrolidine, 1-[[(3-ethoxy-1-adamantyl)amino]acetyl]-2-cyano-, (S)—;
   or, in each case, a pharmaceutically acceptable acid addition salt thereof.

More preferably, the DPP-IV inhibitor of formula (IIIA) or (IIIB) is

2-Pyrrolidinecarbonitrile, 1-[[(3-hydroxytricyclo[3.3.1.13,7]dec-1-yl)amino]acetyl]-, (2S)—, or a pharmaceutically acceptable acid addition salt thereof. This compound is also referred to as pyrrolidine, 1-[(3-hydroxy-1-adamantyl)amino]acetyl-2cyano-, (S), or (S)-1-[2-((5S,7S)-3-Hydroxy-adamantan-1-ylamino)-acetyl]-pyrrolidine-2-carbonitrile, or Vildagliptin. All of the above mentioned specific DPP-IV inhibitors of formula (IIIA) or (IIIB) have been disclosed and described in WO00/034241.

In addition, in the pharmaceutical compositions according to the present invention, the DPP-IV inhibitor can preferably be a compound of formula (IV)

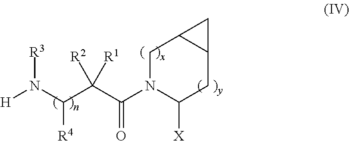

wherein x is 0 or 1 and y is 0 or 1, provided that
x=1 when y=0 and
x=0 when y=1; and wherein
n is 0 or 1;

### X is H or CN;

R¹, R², R³ and R⁴ are the same or different and are independently selected from hydrogen, alkyl, alkenyl, alkynyl, cycloalkyl, cycloalkylalkyl, bicycloalkyl, tricycloalkyl, alkylcycloalkyl, hydroxyalkyl, hydroxyalkylcycloalkyl, hydroxycycloalkyl, hydroxybicycloalkyl, hydroxytricycloalkyl, bicycloalkylalkyl, alkylthioalkyl, arylalkylthioalkyl, cycloalkenyl, aryl, aralkyl, heteroaryl, heteroarylalkyl, cycloheteroalkyl or cycloheteroalkylalkyl; all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, polycycloalkyl, heteroarylamino, arylamino, cycloheteroalkyl, cycloheteroalkylalkyl, hydroxy, hydroxyalkyl, nitro, cyano, amino, substituted amino, alkylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkylsulfonylamino, alkylaminocarbonylamino, alkoxycarbonylamino, alkylsulfonyl, aminosulfinyl, aminosulfonyl, alkylsulfinyl, sulfonamido or sulfonyl; and R¹ and R³ may optionally be taken together to form —(CR⁵R⁶)ₘ— where m is 2 to 6, and R⁵ and R⁶ are the same or different and are independently selected from hydroxy, alkoxy, H, alkyl, alkenyl, alkynyl, cycloalkyl, halo, amino, substituted amino, cycloalkylalkyl, cycloalkenyl, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino, or R¹ and R⁴ may optionally be taken together to form —(CR⁷R⁸)ₚ— wherein p is 2 to 6, and
R⁷ and R⁸ are the same or different and are independently selected from hydroxy, alkoxy, cyano, H, alkyl, alkenyl, alkynyl, cycloalkyl, cycloalkylalkyl, cycloalkenyl, halo, amino, substituted amino, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino, or optionally R¹ and R³ together with

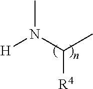

form a 5 to 7 membered ring containing a total of 2 to 4 heteroatoms selected from N, O, S, SO, or SO₂; or optionally R¹ and R³ together with

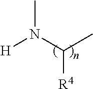

form a 4 to 8 membered cycloheteroalkyl ring wherein the cycloheteroalkyl ring has an optional aryl ring fused thereto or an optional 3 to 7 membered cycloalkyl ring fused thereto; including all stereoisomers thereof;
and a pharmaceutically acceptable salt thereof, or a prodrug ester thereof, and all stereoisomers thereof.

Of the DPP-IV inhibitors of formula (IV), those are preferred, wherein R³ is H, R¹ is H, alkyl, cycloalkyl, bicycloalkyl, tricycloalkyl, alkylcycloalkyl, hydroxyalkyl, hydroxyalkylcycloalkyl, hydroxycycloalkyl hydroxybicycloalkyl, or hydroxytricycloalkyl, R² is H or alkyl, n is 0, X is CN.

The DPP-IV inhibitors of formula (IV) have been disclosed and described in detail in WO01/68603.

Preferably, the DPP-IV inhibitor of formula (IV) is selected from the compounds specifically described in WO01/68603.

More preferably, the DPP-IV inhibitor of formula (IV) is

2-Azabicyclo[3.1.0]hexane-3-carbonitrile, 2-[(2S)-amino(3-hydroxytricyclo[3.3.1.13,7]dec-1-yl)acetyl]-, (1S,3S,5S)—, or a pharmaceutically acceptable acid addition salt thereof. This compound is also referred to as (1S,3S,5S)-2-[(S)-2-Amino-2-(3-hydroxy-adamantan-1-yl)-acetyl]-2-aza-bicyclo[3.1.0]hexane-3-carbonitrile, or Saxagliptin. All of the above mentioned specific DPP-IV inhibitors of formula (IV) have been disclosed and described in WO01/68603.

In addition, in the pharmaceutical compositions according to the present invention, the DPP-IV inhibitor can preferably be a compound of formula (V)

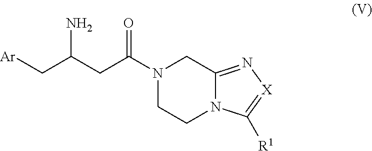

Ar is phenyl which is unsubstituted or substituted with 1-5 of R³, wherein R³ is independently selected from the group consisting of:
halogen,
(2) C₁₋₆ alkyl, which is linear or branched and is unsubstituted or substituted with 1-5 halogens,
(3) OC₁₋₆ alkyl, which is linear or branched and is unsubstituted or substituted with 1-5 halogens, and

### (4) CN;

X is selected from the group consisting of:

### N, and

### (2) CR²;

R¹ and R² are independently selected from the group consisting of:
hydrogen,

### (2) CN,

(3) C₁₋₁₀ alkyl, which is linear or branched and which is unsubstituted or substituted with 1-5 halogens or phenyl, which is unsubstituted or substituted with 1-5 substituents independently selected from halogen, CN, OH, R⁴, OR⁴, NHSO₂R⁴, SO₂R⁴, CO₂H, and CO₂C₁₋₆alkyl, wherein the CO₂C₁₋₆ alkyl is linear or branched,
(4) phenyl which is unsubstituted or substituted with 1-5 substituents independently selected from halogen, CN, OH, R⁴, OR⁴, NHSO₂R⁴, SO₂R⁴, CO₂H, and CO₂C₁₋₆alkyl, wherein the CO₂C₁₋₆alkyl is linear or branched, and
(5) a 5- or 6-membered heterocycle which may be saturated or unsaturated comprising 1-4 heteroatoms independently selected from N, S and O, the heterocycle being unsubstituted or substituted with 1-3 substituents independently selected from oxo, OH, halogen, C₁₋₆alkyl, and OC₁₋₆alkyl, wherein the C₁₋₆alkyl and OC₁₋₆alkyl are linear or branched and optionally substituted with 1-5 halogens;
R⁴ is C₁₋₆alkyl, which is linear or branched and which is unsubstituted or substituted with 1-5 groups independently selected from halogen, CO₂H, and CO₂C₁₋₆alkyl, wherein the CO₂C₁₋₆alkyl is linear or branched;
and pharmaceutically acceptable salts thereof and individual diastereomers thereof.

The DPP-IV inhibitors of formula (V) have been disclosed and described in detail in WO03/004498.

Preferably, the DPP-IV inhibitor of formula (V) is selected from the compounds specifically described in WO03/004498.

More preferably, the DPP-IV inhibitor of formula (V) is

1,2,4-Triazolo[4,3-a]pyrazine, 7-[(3R)-3-amino-1-oxo-4-(2,4,5-trifluorophenyebutyl]-5,6,7,8-tetrahydro-3-(trifluoromethyl)-, and pharmaceutically acceptable salts thereof, preferably 1,2,4-Triazolo[4,3-a]pyrazine, 7-[(3R)-3-amino-1-oxo-4-(2,4,5-trifluorophenyebutyl]-5,6,7,8-tetrahydro-3-(trifluoromethyl)-, phosphate (1:1). This compound is also referred to as (R)-3-Amino-1-(3-trifluoromethyl-5,6-dihydro-8H-[1,2,4]triazolo[4,3-a]pyrazin-7-yl)-4-(2,4,5-trifluoro-phenyl)-butan-1-one, or Sitagliptin and has been disclosed and described in WO03/004498.

Particularly preferred is the above described pharmaceutical composition, wherein the DPP-IV inhibitor is selected from the group consisting of
(2S)-1-{[2-(5-Methyl-2-phenyl-oxazol-4-yl)-ethylamino]-acetyl}-pyrrolidine-2-carbonitrile,
(2S)-1-{[1,1-Dimethyl-3-(4-pyridin-3-yl-imidazol-1-yl)-propylamino]-acetyl}-pyrrolidine-2-carbonitrile,
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one,
(S,S,S,S)-1-(2-Amino-9,10-dimethoxy-1,3,4,6,7,11b-hexahydro-2H-pyrido[2,1-a]isoquinolin-3-yl)-4-methyl-pyrrolidin-2-one,
(S)-1-[2-((5S,7S)-3-Hydroxy-adamantan-1-ylamino)-acetyl]-pyrrolidine-2-carbonitrile,
(1S,3S,5S)-2-[(S)-2-Amino-2-(3-hydroxy-adamantan-1-yl)-acetyl]-2-aza-bicyclo[3.1.0]hexane-3-carbonitrile, and
(R)-3-Amino-1-(3-trifluoromethyl-5,6-dihydro-8H-[1,2,4]triazolo[4,3-a]pyrazin-7-yl)-4-(2,4,5-trifluoro-phenyl)-butan-1-one,
   and pharmaceutically acceptable salts thereof.

In a more preferred embodiment, the DPP-IV inhibitor is (2S)-1-{[2-(5-Methyl-2-phenyl-oxazol-4-yl)-ethylamino]-acetyl}-pyrrolidine-2-carbonitrile, or a pharmaceutically acceptable salt thereof, more preferably the mesylate.

In another more preferred embodiment, the DPP-IV inhibitor is (2S)-1-{[1,1-Dimethyl-3-(4-pyridin-3-yl-imidazol-1-yl)-propylamino]-acetyl}-pyrrolidine-2-carbonitrile, or a pharmaceutically acceptable salt thereof.

In another more preferred embodiment, the DPP-IV inhibitor is (S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one, or a pharmaceutically acceptable salt thereof.

In another more preferred embodiment, the DPP-IV inhibitor is (S,S,S,S)-1-(2-Amino-9,10-dimethoxy-1,3,4,6,7,11b-hexahydro-2H-pyrido[2,1-a]isoquinolin-3-yl)-4-methyl-pyrrolidin-2-one, or a pharmaceutically acceptable salt thereof.

In another more preferred embodiment, the DPP-IV inhibitor is (S)-1-[2-((5S,7S)-3-Hydroxy-adamantan-1-ylamino)-acetyl]-pyrrolidine-2-carbonitrile, or a pharmaceutically acceptable salt thereof.

In another more preferred embodiment, the DPP-IV inhibitor is (1S,3S,5S)-2-[(S)-2-Amino-2-(3-hydroxy-adamantan-1-yl)-acetyl]-2-aza-bicyclo[3.1.0]hexane-3-carbonitrile, or a pharmaceutically acceptable salt thereof.

In another more preferred embodiment, the DPP-IV inhibitor is (R)-3-Amino-1-(3-trifluoromethyl-5,6-dihydro-8H-[1,2,4]triazolo[4,3-a]pyrazin-7-yl)-4-(2,4,5-trifluoro-phenyl)-butan-1-one, or a pharmaceutically acceptable salts thereof.

(2S)-1-{[2-(5-Methyl-2-phenyl-oxazol-4-yl)-ethylamino]-acetyl}-pyrrolidine-2-carbonitrile is preferably used in form of the mesylate salt.

(R)-3-Amino-1-(3-trifluoromethyl-5,6-dihydro-8H-[1,2,4]triazolo[4,3-a]pyrazin-7-yl)-4-(2,4,5-trifluoro-phenyl)-butan-1-one is preferably used in the form of the phosphate salt.

Unless otherwise indicated, the meaning and scope of the various terms used to describe the DPP-IV inhibitors above are the same as disclosed in WO 03/037327, WO 2005/000848, WO00/34241, WO01/68603 and WO03/004498 respectively. The terms can e.g. have the following meanings.

The term “lower” is used to mean a group consisting of one to seven, one to six, preferably of one to four carbon atom(s).

The term “halogen” refers to fluorine, chlorine, bromine and iodine, preferably to fluorine, bromine and chlorine, more preferably to fluorine and chlorine. Most preferred halogen is fluorine.

The term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms. Alkyl groups can optionally be substituted e.g. with halogen, hydroxy, lower-alkoxy, lower-alkoxy-carbonyl, NH₂, N(H, lower-alkyl) and/or N(lower-alkyl)₂. Unsubstituted alkyl groups are preferred.

The term “lower-alkyl”, alone or in combination with other groups, refers to a branched or straight-chain monovalent alkyl radical of one to six or one to seven carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like. Preferable lower alkyl residues are methyl and ethyl, with methyl being especially preferred. A lower-alkyl group may optionally have a substitution pattern as described earlier in connection with the term “alkyl”. Unsubstituted lower-alkyl groups are preferred.

The term “alkoxy” refers to the group R′—O—, wherein R′ is alkyl. The term “lower-alkoxy” refers to the group R′—O—, wherein R′ is lower-alkyl. Examples of lower-alkoxy groups are e.g. methoxy, ethoxy, propoxy, isopropoxy, butoxy, isobutoxy and hexyloxy. Alkoxy and lower-alkoxy groups may optionally have a substitution pattern as described earlier in connection with the term “alkyl”. Unsubstituted alkoxy and lower-alkoxy groups are preferred.

The term “halogenated lower alkyl” refers to a lower alkyl group wherein at least one of the hydrogens of the lower alkyl group is replaced by a halogen atom, preferably fluoro or chloro, most preferably fluoro. Among the preferred halogenated lower alkyl groups are trifluoromethyl, difluoromethyl, fluoromethyl and chloromethyl, with fluoromethyl being especially preferred.

The term “lower alkoxycarbonyl” refers to the group R′—O—C(O)—, wherein R′ is lower alkyl.

The term “cycloalkyl” refers to a monovalent carbocyclic radical of three to six, preferably three to five carbon atoms. This term is further exemplified by radicals such as cyclopropyl, cyclobutyl, cyclopentyl and cyclohexyl, with cyclopropyl and cyclobutyl being preferred. Such cycloalkyl residues may optionally be mono-, di- or tri-substituted, independently, by lower alkyl or by halogen.

The term “aryl” relates to the phenyl or naphthyl group, preferably the phenyl group, which can optionally be mono- or multiply-substituted by lower-alkyl, lower-alkoxy, halogen, CN, CF₃, hydroxy, NO₂, NH₂, N(H, lower-alkyl), N(lower-alkyl)₂, carboxy, aminocarbonyl, phenyl, benzyl, phenoxy, and/or benzyloxy. Preferred substituents are lower-alkyl, lower-alkoxy, halogen, CN, and/or CF₃. The term “aryl” can also refer to an aromatic monovalent mono- or polycarbocyclic radical, such as phenyl or naphthyl, preferably phenyl, which may optionally be mono-, di- or tri-substituted, independently, by lower alkyl, lower alkoxy, halo, cyano, azido, amino, di-lower alkyl amino or hydroxy.

The term “heteroaryl” refers to an aromatic 5- or 6-membered ring which can comprise 1, 2 or 3 atoms selected from nitrogen, oxygen and/or sulphur such as furyl, pyrrolyl, pyridyl, 1,2-, 1,3- and 1,4-diazinyl, thienyl, oxazolyl, oxadiazolyl, isoxazolyl, thiazolyl, isothiazolyl or imidazolyl. A heteroaryl group may optionally have a substitution pattern as described earlier in connection with the term “aryl”.

The term “5-membered heteroaryl” refers to an aromatic 5-membered ring which can comprise 1 to 4 atoms selected from nitrogen, oxygen and/or sulphur such as furyl, thienyl, pyrrolyl, imidazolyl, pyrazolyl, oxazolyl, isoxazolyl, thiazolyl, isothiazolyl, oxadiazolyl such as 1,3,4- and 1,2,4-oxadiazolyl, triazolyl or tetrazolyl. Preferred 5-membered heteroaryl groups are oxazolyl, imidazolyl, pyrazolyl, triazolyl, 1,3,4- and 1,2,4-oxadiazolyl and thiazolyl. A 5-membered heteroaryl group can optionally be substituted with lower-alkyl, lower-alkoxy, halogen, CN, CF₃, trifluoroacetyl, aryl, heteroaryl, and carbonyl, which carbonyl group can optionally be substituted with lower-alkyl, lower-alkoxy, halogen, CN, CF₃, aryl, or heteroaryl.

The term “a 4-, 5-, 6- or 7-membered saturated or unsaturated heterocyclic ring optionally containing a further heteroatom selected from nitrogen, oxygen and sulfur” refers to a non-aromatic heterocyclic ring, said heterocyclic ring being optionally mono-, di-, or tri-substituted, independently, with lower alkyl, halogenated lower alkyl, oxo, dioxo and/or cyano. Such saturated heterocyclic rings are for example pyrrolidinyl, piperidinyl, azepanyl, [1,2]thiazinanyl, [1,3]oxazinanyl, oxazolidinyl, thiazolidinyl or azetidinyl. Examples of such unsaturated heterocyclic rings are 5,6-dihydro-1H-pyridin-2-one, pyrrolinyl, tetrahydropyridine or dihydropyridine.

The term “heterocyclyl” refers to a 5- or 6-membered aromatic or saturated N-heterocyclic residue, which may optionally contain a further nitrogen or oxygen atom, such as imidazolyl, pyrazolyl, thiazolyl, pyridyl, pyrimidyl, morpholino, piperazino, piperidino or pyrrolidino, preferably pyridyl, thiazolyl or morpholino. Such heterocyclic rings may optionally be mono-, di- or tri-substituted, independently, by lower alkyl, lower alkoxy, halo, cyano, azido, amino, di-lower alkyl amino or hydroxy. Preferable substituent is lower alkyl, with methyl being preferred.

The term “monocyclic heterocyclyl” refers to non aromatic monocyclic heterocycles with 5 or 6 ring members, which comprise 1, 2 or 3 hetero atoms selected from nitrogen, oxygen and sulfur. Examples of suitable monocyclic heterocyclyl groups are piperidinyl and morpholinyl. A monocyclic heterocyclyl may be substituted with lower-alkyl.

The term “bi- or tricyclic heterocyclyl” refers to bicyclic or tricyclic aromatic groups comprising two or three 5- or 6-membered rings, in which one or more rings can comprise 1, 2 or 3 atoms selected from nitrogen, oxygen and/or sulphur, and which can be partially hydrogenated.

Examples of bi- or tricyclic heterocyclyl groups are e.g. indolyl, aza-indolyl such as 2-, 3-, 4-, 5-, 6- or 7-aza-indolyl, indolinyl carbazolyl, benzothiophenyl, benzothiazolyl, benzooxazolyl, benzimidazolyl, 4,5,6,7-tetrahydro-thiazolo[5,4-c]pyridinyl, 4,5,6,7-tetrahydro-benzthiazolyl, 8H-indeno[1,2-d]thiazolyl and quinolinyl. Preferred bi- or tricyclic heterocyclyl groups are benzothiazolyl and 4,5,6,7-tetrahydro-thiazolo[5,4-c]pyridinyl. A bi- or tricyclic heterocyclyl group can optionally have a substitution pattern as described earlier in connection with the term “5-membered heteroaryl”.

The term “pharmaceutically acceptable salts” embraces salts of the compounds of formula (I) with inorganic or organic acids such as hydrochloric acid, hydrobromic acid, nitric acid, sulphuric acid, phosphoric acid, citric acid, formic acid, maleic acid, acetic acid, fumaric acid, succinic acid, tartaric acid, methanesulphonic acid, salicylic acid, p-toluenesulphonic acid and the like, which are non toxic to living organisms. Preferred salts with acids are formates, maleates, citrates, hydrochlorides, hydrobromides and methanesulfonic acid salts, with hydrochlorides being especially preferred.

A preferred embodiment of the present invention relates to a pharmaceutical composition as defined above, additionally comprising a DPP-IV inhibitor which is released in the stomach or upper gut. A release in the stomach or upper gut in combination with a release in the lower gastrointestinal tract or ileum has the potential of synergistic effects between the local effects of the two sections. Release in the duodenum does not have a beneficial effect. Preferred is a pharmaceutical composition as defined above, wherein 40 to 60% of the DPP-IV inhibitor is released in the stomach or upper gut and 40 to 60% of the DPP-IV inhibitor is released in the lower gastrointestinal tract. In the pharmaceutical composition described above, the DPP-IV inhibitor is preferably not released in the duodenum. In a particularly preferred embodiment of the present invention, the pharmaceutical composition described above is a two layer tablet. In such two layer tablets a DPP-IV inhibitor, which is present in the first layer, is released in the stomach or upper gut. The second layer, which can comprise an adequate coating as described before, comprises the DPP-IV inhibitor which is released in the lower gastrointestinal tract or ileum, preferably the ileum. A pharmaceutical composition as described above can also constitute of two separate units, one unit releasing the DPP-IV inhibitor in the stomach or upper gut and one unit which releases the DPP-IV inhibitor in the lower gastrointestinal tract, preferably the ileum. In analogy, pharmaceutical compositions as described above can also be mixtures of different, optionally coated, pellets or minitablets, applied in a single capsule or mixed with additional excipients and compressed to tablets.

Another preferred embodiment of the present invention relates to the use of a DPP-IV inhibitor for the preparation of a pharmaceutical composition as defined above for the treatment of diseases associated with elevated blood glucose levels. Preferably, the disease associated with elevated blood glucose levels is diabetes mellitus, type I diabetes, type II diabetes, diabetes secondary to pancreatic disease, diabetes related to steroid use, type III diabetes, hyperglycaemia, diabetic complications or insulin resistance more preferably type II diabetes.

A further preferred embodiment of the present invention relates to a method for the treatment of diseases associated with elevated blood glucose levels, preferably diabetes mellitus, type I diabetes, type II diabetes, diabetes secondary to pancreatic disease, diabetes related to steroid use, type III diabetes, hyperglycaemia, diabetic complications or insulin resistance, particularly type II diabetes, which method comprises administering a pharmaceutical composition as defined above to a human being or animal.

The compositions of the present invention may be formulated in a conventional manner using one or more pharmaceutically acceptable carriers. The pharmaceutical compositions of the present invention are preferably for oral administration.

For oral administration, the pharmaceutical compositions may take the form of, for example, tablets, minitablets, pellets or capsules prepared by conventional means with pharmaceutically acceptable excipients such as binding agents (e.g. pregelatinised maize starch, polyvinylpyrrolidone, polyvinylacetate or hydroxypropylmethylcellulose); fillers (e.g. lactose, microcrystalline cellulose or calcium phosphate); lubricants (e.g. magnesium stearate Sodium stearyl fumarate, glyceryl behenate, Sotalc or silica); disintegrants (e.g. potato starch or sodium starch glycollate); or wetting agents (e.g. sodium lauryl sulfate), binders (e.g. Crospovidone, N-methylpyrrolidone). In order to achieve a release of the active compound, namely the DPP-IV inhibitor, in the ileum, appropriate coatings can be used, such as coats of esters and ethers of methacrylic acid and copolymers thereof. The coatings may be applied by conventional methods such as fluid bed coating or pan coating on tablets or capsules, as well as on pellets or minitablets. A suitable subcoat may also be applied. Such a coat could base e.g. on polyvinylacetate, hydroxypropylmethylcellulose, Ethylcellulose other derivatives of cellulose or mixtures thereof.

A proposed dose of the DPP-IV inhibitor in the pharmaceutical compositions of the present invention to be administered to the average adult human for the treatment of the conditions referred to above (e.g. type II diabetes) can e.g. be in the range of 10 to 1000 mg of the active ingredient per unit dose, more preferably 10 to 400 mg per unit dose, more preferably 100 to 400 mg per unit dose, which could be administered, for example, 1 to 2 times per day.

### Assay Procedures

The following tests can be carried out in order to determine the biological activity of DPP-IV inhibitors.

Activity of DPP-IV inhibitors are tested with natural human DPP-IV derived from a human plasma pool or with recombinant human DPP-IV. Human citrate plasma from different donors is pooled, filtered through a 0.2 micron membrane under sterile conditions and aliquots of 1 ml are shock frozen and stored at −120° C. until used. In the colorimetric DPP-IV assay 5 to 10 μl human plasma and in the fluorometric assay 1.0 μl of human plasma in a total assay volume of 100 μl is used as an enzyme source. The cDNA of the human DPP-IV sequence of amino acid 31- to 766, restricted for the N-terminus and the transmembrane domain, is cloned into pichia pastoris. Human DPP-IV is expressed and purified from the culture medium using conventional column chromatography including size exclusion and anion and cation chromatography. The purity of the final enzyme preparation of Coomassie blue SDS-PAGE is >95%. In the colorimetric DPP-IV assay 20 ng rec.-h DPP-IV and in the fluorometric assay 2 ng rec-h DPP-IV in a total assay volume of 100 μl is used as an enzyme source.

In the fluorogenic assay Ala-Pro-7-amido-4-trifluoromethylcoumarin (Calbiochem No 125510) is used as a substrate. A 20 mM stock solution in 10% DMF/H₂O is stored at −20° C. until use. In IC50 determinations a final substrate concentration of 50 μM is used. In assays to determine kinetic parameters as Km, Vmax, Ki, the substrate concentration is varied between 10 μM and 500 μM.

In the colorimetric assay H-Ala-Pro-pNA.HCl (Bachem L-1115) is used as a substrate. A 10 mM stock solution in 10% MeOH/H₂O is stored at −20° C. until use. In IC50 determinations a final substrate concentration of 200 μM is used. In assays to determine kinetic parameters as Km, Vmax, Ki, the substrate concentration is varied between 100 μM and 2000 μM. Fluorescence is detected in a Perkin Elmer Luminescence Spectrometer LS 50B at an excitation wavelength of 400 nm and an emission wavelength of 505 nm continuously every 15 seconds for 10 to 30 minutes. Initial rate constants are calculated by best fit linear regression. The absorption of pNA liberated from the colorimetric substrate is detected in a Packard SpectraCount at 405 nM continuously every 2 minutes for 30 to 120 minutes. Initial rate constants are calculated by best fit linear regression.

DPP-IV activity assays are performed in 96 well plates at 37° C. in a total assay volume of 100 μl. The assay buffer consists of 50 mM Tris/HCl pH 7.8 containing 0.1 mg/ml BSA and 100 mM NaCl. Test compounds are solved in 100% DMSO, diluted to the desired concentration in 10% DMSO/H₂O. The final DMSO concentration in the assay is 1% (v/v). At this concentration enzyme inactivation by DMSO is <5%. Compounds are with (10 minutes at 37° C.) and without preincubation with the enzyme. Enzyme reactions are started with substrate application followed by immediate mixing.

IC₅₀ determinations of test compounds are calculated by non-linear best fit regression of the DPP-IV inhibition of at least 5 different compound concentrations. Kinetic parameters of the enzyme reaction are calculated at least 5 different substrate concentrations and at least 5 different test compound concentrations.

DPP-IV inhibitors preferably exhibit a biological activity which can be characterised by an IC₅₀ value below 10 μM, preferably below 1 μM. IC₅₀ values of DPP-IV inhibitors are usually above 0.01 nM, preferably above 0.1 nM.

Such inhibitory activity can be characterised by the IC₅₀ value. A DPP-IV inhibitor preferably exhibits an IC₅₀ value below 10 μM, preferably below 1 μM. IC₅₀ values of DPP-IV inhibitors are usually above 0.01 nM, preferably above 0.1 nM.

### EXAMPLES

### Example 1

Coated tablets with the compositions shown in the table below are made according to standard procedures. The specific DPP-IV inhibitor mentioned in the table can be replaced by other DPP-IV inhibitors mentioned above.

| | | 100 mg | | 200 mg | | 400 mg | |
|---|---|---|---|---|---|---|---|
| Component | Description | tablet | | tablet | | tablet | |
| Granulate | | | | | | | |
| (2S)-1-{[2-(5- | DPPIV | 128.4 | mg | 256.8 | mg | 513.6 | mg |
| Methyl-2-phenyl- | inhibitor | | | | | | |
| oxazol-4-yl)- | | | | | | | |
| ethylamino]- | | | | | | | |
| acetyl}- | | | | | | | |
| pyrrolidine-2- | | | | | | | |
| carbonitrile | | | | | | | |
| mesylate | | | | | | | |
| Microcrystalline | Filler | 56.4 | mg | 112.80 | mg | 225.6 | mg |
| Cellulose | | | | | | | |
| (Avicel PH-101) | | | | | | | |
| Sodium stearyl | Glidant | 0.9625 | mg | 1.925 | mg | 3.85 | mg |
| fumarate | | | | | | | |
| Kernel | | | | | | | |
| (externel phase) | | | | | | | |
| Talc | Anti- | 6 | mg | 9 | mg | 12 | mg |
| | adhesive | | | | | | |
| Sodium stearyl | Glidant/ | 2 | mg | 3 | mg | 4 | mg |
| fumarate | Lubricant | | | | | | |
| Coat | | | | | | | |
| Opadry | Film former | 9.50 | mg | 15.00 | mg | 30.00 | mg |
| Eudragit S 100 | Coat | 15 | mg | 25 | mg | 50 | mg |
| Total: | | 217 | mg | 425 | mg | 850 | mg |

### Example 2

Coated capsules with the compositions shown in the table below are made according to standard procedures. The specific DPP-IV inhibitor mentioned in the table can be replaced by other DPP-IV inhibitors mentioned above.

| | | 50 mg | | 150 mg | |
|---|---|---|---|---|---|
| Component | Description | capsule | | capsule | |
| Granulate | | | | | |
| (2S)-1-{[1,1-Dimethyl-3- | DPP-IV | 50 | mg | 150 | mg |
| (4-pyridin-3-yl-imidazol-1- | inhibitor | | | | |
| yl)-propylamino]-acetyl}- | | | | | |
| pyrrolidine-2-carbonitrile | | | | | |
| Microcrystalline Cellulose | Filler | 56.4 | mg | 112.80 | mg |
| (Avicel PH-102) | | | | | |
| Externel phase | | | | | |
| Talc | Anti- | 1.925 | mg | 3.85 | mg |
| | adhesive | | | | |
| Sodium stearyl fumarate | Glidant/ | 4.8125 | mg | 9.625 | mg |
| | Lubricant | | | | |
| Capsule | | | | | |
| Eudragit S:Eudragit L 25:75 | | 25 | mg | 40 | mg |

### Example 3

Capsules with coated pellets with the compositions shown in the table below are made according to standard procedures. The specific DPP-IV inhibitor mentioned in the table can be replaced by other DPP-IV inhibitors mentioned above.

| | | 50 mg | | 150 mg | |
|---|---|---|---|---|---|
| Component | Description | capsule | | capsule | |
| Granulate | | | | | |
| (S)-1-((2S,3S,11bS)-2-Amino- | DPP-IV | 50 | mg | 150 | mg |
| 9,10-dimethoxy-1,3,4,6,7,11b- | inhibitor | | | | |
| hexahydro-2H-pyrido[2,1-a] | | | | | |
| isoquinolin-3-yl)-4- | | | | | |
| fluoromethyl-pyrrolidin-2-one | | | | | |
| Microcrystalline Cellulose | Filler | 60 | mg | 80 | mg |
| (Avicel PH-102) | | | | | |
| Pregelatinized starch | Binder | 30 | | 50 | |
| Externel phase | | | | | |
| Talc | Anti- | 1.925 | mg | 3.85 | mg |
| | adhesive | | | | |
| Magnesium stearate | Glidant/ | 4.8125 | mg | 9.625 | mg |
| | Lubricant | | | | |
| Coat | | | | | |
| Eudragit L:Eudragit FS 75:25 | | 60 | mg | 100 | mg |
| Capsule | | | | | |

### Example 4

Bi-layer tablets with the compositions shown in the table below are made according to standard procedures. The specific DPP-IV inhibitor mentioned in the table can be replaced by other DPP-IV inhibitors mentioned above.

| | | 100 mg | | 200 mg | | 400 mg | |
|---|---|---|---|---|---|---|---|
| Component | Description | tablet | | tablet | | tablet | |
| Granulate | | | | | | | |
| (S)-1-((2S,3S,11bS)- | DPP-IV | 100 | mg | 200 | mg | 400 | mg |
| 2-Amino-9,10- | inhibitor | | | | | | |
| dimethoxy-1,3,4,6,7, | | | | | | | |
| 11b-hexahydro-2H- | | | | | | | |
| pyrido[2,1-a] | | | | | | | |
| isoquinolin-3-yl)-4- | | | | | | | |
| fluoromethyl- | | | | | | | |
| pyrrolidin-2-one | | | | | | | |
| Microcrystalline | Filler | 56.4 | mg | 112.80 | mg | 225.6 | mg |
| Cellulose | | | | | | | |
| (Avicel PH-101) | | | | | | | |
| Lactose monohydrate | Filler | 10 | | 20 | | 40 | mg |
| Polyvinylpyrrolidone | Binder | 10 | | 20 | | 40 | |
| total | | 176.4 | | 352.8 | | 705.6 | |
| 1st layer | Granulate, | 88.2 | | 176.4 | | 352.8 | |
| (externel phase) | half of total | | | | | | |
| Talc | Anit- | 1 | mg | 2 | mg | 4 | mg |
| | adhesive | | | | | | |
| Glycerol behenate | Glidant/ | 3 | mg | 6 | mg | 12 | mg |
| | Lubricant | | | | | | |
| Coat | Only | | | | | | |
| | around 1^{st} | | | | | | |
| | layer | | | | | | |
| Eudragit S | | 15 | mg | 25 | mg | 50 | mg |
| 2^{nd} layer | Granulate, | 88.2 | | 176.4 | | 352.8 | |
| | half of total | | | | | | |
| Talc | Anit- | 1 | mg | 2 | mg | 4 | mg |
| | adhesive | | | | | | |
| Glycerol behenate | Glidant/ | 6 | mg | 3 | mg | 12 | mg |
| | Lubricant | | | | | | |
| Final coat | Around | | | | | | |
| | total tablet | | | | | | |
| Opadry II | Film former | 9.50 | mg | 15.00 | mg | 30.00 | mg |

### Example 5

A pharmacoscintigraphic evaluation of the regional drug absorption and pharmacodynamics of (2S)-1-{[2-(5-Methyl-2-phenyl-oxazol-4-yl)-ethylamino]-acetyl}-pyrrolidine-2-carbonitrile mesylate in up to 9 healthy male or female volunteers following administration to four different sites of the gastrointestinal tract: stomach, proximal small bowel, ileum and ascending colon was carried out. The study was conducted as an open label, 4-way cross-over design consisting of 4 study periods of approximately 2-3 days duration, each separated by washout period of at least 4 days.

During each study period, 400 mg (2S)-1-{[2-(5-Methyl-2-phenyl-oxazol-4-yl)-ethylamino]-acetyl}-pyrrolidine-2-carbonitrile mesylate was delivered to the appropriate gastrointestinal target using Enterion™ capsule technology. The capsule was administered with water containing a radiolabelled marker (^{99m}Tc-DTPA) which was used to define the gastrointestinal anatomy and the movement of the capsule was followed by means of an ¹¹¹In marker within the device. The location of both radiolabels was monitored on images obtained from a dual wavelength gamma camera. Capsule activation and thereby drug release was achieved by applying an external signal. The release was planned to occur within 5 hours of the administration of a standardised low calorie meal.

The pharmacokinetics of (2S)-1-{[2-(5-Methyl-2-phenyl-oxazol-4-yl)-ethylamino]-acetyl}-pyrrolidine-2-carbonitrile mesylate were determined after each administration by monitoring plasma concentrations of parent drug and metabolites. The pharmacodynamic response was assessed by measuring the concentrations of circulating markers (glucose, insulin, glucagon and GLP-1) for up to 4 hours following an oral glucose tolerance test (OGTT), which itself was carried out 2 hours after release of the drug substance. A control OGTT response (i.e. no drug treatment) was established for each subject before the first treatment period began.

Plasma profiles of (2S)-1-{[2-(5-Methyl-2-phenyl-oxazol-4-yl)-ethylamino]-acetyl}-pyrrolidine-2-carbonitrile mesylate indicated that the absorption and elimination rate was broadly similar for all routes of administration except for the colon, where concentrations were substantially lower but were sustained for a longer period (6-8 hours post dose). Average exposure was slighter greater after delivery to the proximal small bowel (duodenum).

TABLE 1
Mean (SD) Plasma Exposure Parameters

| | Mean (SD) Cₘₐₓ | | Mean (SD) AUC | |
|---|---|---|---|---|
| | (ng/mL) | | (ng · h/mL) | |
| Dosing | standard deviation | | standard deviation | |
| Region | in brackets | | in brackets | |
| Stomach | 5570 | (877) | 12200 | (2560) |
| Duodenum | 7580 | (2410) | 14200 | (5810) |
| Ileum | 5420 | (833) | 12300 | (3580) |
| Colon | 736 | (529) | 3540 | (2760) |

### Pharmacodynamic Response

Mean blood glucose area under the effect curve (AUEC) following an OGTT was substantially decreased vs control following both stomach and ileal delivery of the DPPIV inhibitor. These reductions in blood glucose did not appear to be the result of increased blood insulin levels. However, only ileal delivery gave a sustained systematic increase in the primary mechanistic biomarker, active glucagon-like peptide 1.

TABLE 2
Mean Delta (baseline corrected) Glucose, Insulin
and GLP-1 AUECs Following OGTT Site-Specific
Delivery of DPPIV Inhibitor to Healthy Volunteers

| | Mean Delta Blood | Mean Plasma | Mean Plasma |
|---|---|---|---|
| Dosing | Glucose AUEC | Insulin AUEC | GLP-1 AUEC |
| Region | (% Control) | (% Control) | (% Control) |
| Stomach | 55 | 65 | 151 |
| PSB* | 130 | 160 | 180 |
| Ileum | 47 | 47 | 340 |
| Colon | 83 | 101 | 87 |

### Example 6

A study was conducted in an in-house cynomolgus monkey model, in which permanent cannulae had been surgically attached to various sections of their intestine. This animal model allows compounds to be delivered to precise regions of the intestine in the intact animal in vivo. A single dose cross-over study was performed in three animals, where 5 mg/kg of (2S)-1-{[1,1-Dimethyl-3-(4-pyridin-3-yl-imidazol-1-yl)-propylamino]-acetyl}-pyrrolidine-2-carbonitrile was delivered (with sufficient wash out periods in between), in solution by gavage to the stomach or via the cannulae to the duodenum, the jejunum-ileum junction or the top of the ascending colon, respectively. An oral glucose challenge was performed in each animal for each treatment 2 hours post-dose of compound (plus a pre-study control). Full plasma PK and DPPIV inhibition profiles were obtained for each treatment. Blood glucose profiles were measured for 3 hours post-glucose challenge.

Using this model it was shown with (2S)-1-{[1,1-Dimethyl-3-(4-pyridin-3-yl-imidazol-1-yl)-propylamino]-acetyl}-pyrrolidine-2-carbonitrile that delivery of the compound to the stomach, ileum or the ascending colon produced a reduction in blood glucose compared to control. Delivery to the ileum-jejunum junction or colon produced both the highest effect on glucose while achieving the lowest systemic exposure to the compound and lowest average plasma DPPIV inhibition. This result demonstrates that the observed efficacy is mainly due to local intestinal effects caused by site-specific delivery of the compound, rather than the action of the DPP-IV inhibitor in the systemic circulation.

TABLE 3
Key Summary PK and PD Parameters for (2S)-1-{[1,1-
Dimethyl-3-(4-pyridin-3-yl-imidazol-1-yl)-propylamino]-
acetyl}-pyrrolidine-2-carbonitrile following absorption site-
specific Delivery on a triple-cannulated monkey model

| | | Mean Plasma | Mean Delta |
|---|---|---|---|
| Dosing | Mean Plasma AUC | DPPIV Inhibition | Glucose AUEC |
| Region | (ng · h/ml) | (% baseline) | (% Control) |
| Stomach | 2280 | 65 | 88 |
| Duodenum | 3890 | 60 | 116 |
| Ileum-jejunum | 1350 | 45 | 76 |
| junction | | | |
| Ascending | 354 | 45 | 52 |
| colon | | | |

It is to be understood that the invention is not limited to the particular embodiments of the invention described above, as variations of the particular embodiments may be made and still fall within the scope of the appended claims.

What is claimed is:

1. A pharmaceutical composition comprising a therapeutically effective amount of a DPP-IV inhibitor, wherein the DPP-IV inhibitor is released in the lower gastrointestinal tract.

2. The pharmaceutical composition according to claim 1, wherein the DPP-IV inhibitor is released in the ileum.

3. The pharmaceutical composition according to claim 1, wherein the DPP-IV inhibitor is released at a pH above 7.0.

4. The pharmaceutical composition according to claim 1, wherein the composition comprises a coating.

5. The pharmaceutical composition according to claim 1, wherein the composition is a tablet or a capsule.

6. The pharmaceutical composition according to claim 5, wherein the tablet or capsule comprises a coating.

7. The pharmaceutical composition according to claim 5, wherein the tablet or capsule comprises coated pellets.

8. The pharmaceutical composition according to claim 1, wherein at least 80% of the DPP-IV inhibitor is released in the lower gastrointestinal tract.

9. The pharmaceutical composition according to claim 1, wherein the DPP-IV inhibitor is released with a delay of 30 to 60 minutes at pH 7.0.

10. The pharmaceutical composition according to claim 1, comprising 10 to 1000 mg of the DPP-IV inhibitor.

11. The pharmaceutical composition according to claim 1, comprising 100 to 400 mg of the DPP-IV inhibitor.

12. The pharmaceutical composition according to claim 1, wherein the DPP-IV inhibitor exhibits a biological activity with an IC₅₀ value below 10 μM.

13. The pharmaceutical composition according to claim 1, wherein the DPP-IV inhibitor is a compound of formula (I) 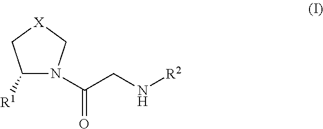
wherein
R¹ is H or CN,
R² is —C(R³,R⁴)—(CH₂)ₙ—R⁵, —C(R³,R⁴)—CH₂—NH—R⁶, —C(R³,R⁴)—CH₂—O—R⁷; or
tetralinyl, tetrahydroquinolinyl or tetrahydroisoquinolinyl, which tetralinyl, tetrahydroquinolinyl or tetrahydroisoquinolinyl group can optionally be substituted with 1 to 3 substituents independently selected from the group consisting of lower-alkyl, lower-alkoxy, halogen, CN, and CF₃,
R³ is hydrogen, lower-alkyl, benzyl, hydroxybenzyl or indolylmethylene,
R⁴ is hydrogen or lower-alkyl, or
R³ and R⁴ are bonded to each other to form a ring together with the carbon atom to which they are attached and —R³—R⁴— is —(CH₂)₂₋₅—,
R⁵ is 5-membered heteroaryl, bi- or tricyclic heterocyclyl, or aminophenyl; optionally substituted with 1 to 3 substituents independently selected from the group consisting of lower-alkyl, lower-alkoxy, halogen, CN, CF₃, trifluoroacetyl, thiophenyl, phenyl, heteroaryl and monocyclic heterocyclyl, which phenyl, heteroaryl or monocyclic heterocyclyl can optionally be substituted with 1 to 3 substituents independently selected from the group consisting of lower-alkyl, lower-alkoxy, benzyloxy, halogen, CF₃, CF₃—O, CN and NH—CO-lower-alkyl,
R⁶ is a) pyridinyl or pyrimidinyl, which is substituted with 1 to 3 substituents independently selected from the group consisting of aryl and heteroaryl, which aryl or heteroaryl group can optionally be substituted with 1 to 3 substituents independently selected from the group consisting of lower-alkyl, lower-alkoxy, halogen, CN, and CF₃,
or b) 5-membered heteroaryl or bi- or tricyclic heterocyclyl, which 5-membered heteroaryl or bi- or tricyclic heterocyclyl can optionally be substituted with 1 to 3 substituents independently selected from the group consisting of lower-alkyl, carbonyl, aryl and heteroaryl, which aryl or heteroaryl group can optionally be substituted with 1 to 3 substituents independently selected from the group consisting of lower-alkyl, lower-alkoxy, halogen, CN, and CF₃, and which carbonyl group can optionally be substituted with lower-alkyl, lower-alkoxy, halogen, CN, CF₃, aryl, or heteroaryl, which aryl or heteroaryl group can optionally be substituted with 1 to 3 substituents independently selected from the group consisting of lower-alkyl, lower-alkoxy, halogen, CN, and CF₃,
R⁷ is aminophenyl, naphthyl or quinolinyl, optionally substituted with 1 to 3 substituents independently selected from the group consisting of lower-alkyl, lower-alkoxy, halogen, CN and CF₃,
X is C(R⁸,R⁹) or S,
R⁸ and R⁹ independently from each other are H or lower-alkyl,
n is 0, 1 or 2,
and pharmaceutically acceptable salts thereof.

14. The pharmaceutical composition according to claim 1, wherein the DPP-IV inhibitor is a compound of formula (II), 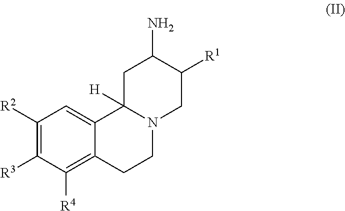
wherein
R¹ is —C(O)—N(R⁵)R⁶ or —N(R⁵)R⁶;
R², R³ and R⁴ are each independently hydrogen, halogen, hydroxy, lower alkyl, lower alkoxy or lower alkenyl, wherein lower alkyl, lower alkoxy and lower alkenyl may optionally be substituted by lower alkoxycarbonyl, aryl or heterocyclyl;
R⁵ is hydrogen, lower alkyl, halogenated lower alkyl or cycloalkyl;
R⁶ is lower alkylsulfonyl, halogenated lower alkylsulfonyl, cycloalkylsulfonyl, lower alkylcarbonyl, halogenated lower alkylcarbonyl, cycloalkylcarbonyl; or
R⁵ and R⁶ together with the nitrogen atom to which they are attached form a 4-, 5-, 6- or 7-membered saturated or unsaturated heterocyclic ring optionally containing a further heteroatom selected from nitrogen, oxygen and sulfur, said heterocyclic ring being optionally mono-, di-, or tri-substituted, independently, with lower alkyl, halogenated lower alkyl, oxo, dioxo and/or cyano;
and pharmaceutically acceptable salts thereof.

15. The pharmaceutical composition according to claim 1, wherein the DPP-IV inhibitor is a compound of formula (IIIA) or (IIIB) 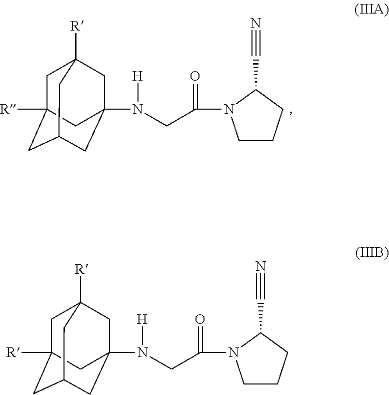
wherein R′ represents hydroxy, C₁-C₇alkoxy, C₁-C₈-alkanoyloxy, or R₅R₄N—CO—O—, where R₄ and R₅ independently are C₁-C₇alkyl or phenyl which is unsubstituted or substituted by a substitutent selected from C₁-C₇alkyl, C₁-C₇alkoxy, halogen and trifluoromethyl and where R₄ additionally is hydrogen; or R₄ and R₅ together represent C₃-C₆ alkylene; and R″ represents hydrogen; or R′ and R″ independently represent C₁-C₇ alkyl; in free form or in form of a pharmaceutically acceptable acid addition salt.

16. The pharmaceutical composition according to claim 1, wherein the DPP-IV inhibitor is a compound of formula (IV) 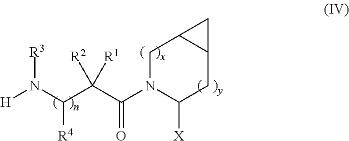
wherein x is 0 or 1 and y is 0 or 1, provided that
x=1 when y=0 and
x=0 when y=1; and wherein
n is 0 or 1;
X is H or CN;
R¹, R², R³ and R⁴ are the same or different and are independently selected from hydrogen, alkyl, alkenyl, alkynyl, cycloalkyl, cycloalkylalkyl, bicycloalkyl, tricycloalkyl, alkylcycloalkyl, hydroxyalkyl, hydroxyalkylcycloalkyl, hydroxycycloalkyl, hydroxybicycloalkyl, hydroxytricycloalkyl, bicycloalkylalkyl, alkylthioalkyl, arylalkylthioalkyl, cycloalkenyl, aryl, aralkyl, heteroaryl, heteroarylalkyl, cycloheteroalkyl or cycloheteroalkylalkyl; all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, polycycloalkyl, heteroarylamino, arylamino, cycloheteroalkyl, cycloheteroalkylalkyl, hydroxy, hydroxyalkyl, nitro, cyano, amino, substituted amino, alkylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkylsulfonylamino, alkylaminocarbonylamino, alkoxycarbonylamino, alkylsulfonyl, aminosulfinyl, aminosulfonyl, alkylsulfinyl, sulfonamido or sulfonyl; and R¹ and R³ may optionally be taken together to form —(CR⁵R⁶)ₘ— where m is 2 to 6, and R⁵ and R⁶ are the same or different and are independently selected from hydroxy, alkoxy, H, alkyl, alkenyl, alkynyl, cycloalkyl, halo, amino, substituted amino, cycloalkylalkyl, cycloalkenyl, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino, or R¹ and R⁴ may optionally be taken together to form —(CR⁷R⁸)ₚ— wherein p is 2 to 6, and
R⁷ and R⁸ are the same or different and are independently selected from hydroxy, alkoxy, cyano, H, alkyl, alkenyl, alkynyl, cycloalkyl, cycloalkylalkyl, cycloalkenyl, halo, amino, substituted amino, aryl, arylalkyl, heteroaryl, heteroarylalkyl, cycloheteroalkyl, cycloheteroalkylalkyl, alkylcarbonylamino, arylcarbonylamino, alkoxycarbonylamino, aryloxycarbonylamino, alkoxycarbonyl, aryloxycarbonyl, or alkylaminocarbonylamino, or optionally R¹ and R³ together with 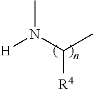
form a 5 to 7 membered ring containing a total of 2 to 4 heteroatoms selected from N, O, S, SO, or SO₂; or optionally R¹ and R³ together with 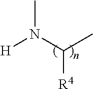
form a 4 to 8 membered cycloheteroalkyl ring wherein the cycloheteroalkyl ring has an optional aryl ring fused thereto or an optional 3 to 7 membered cycloalkyl ring fused thereto;
including all stereoisomers thereof;
and a pharmaceutically acceptable salt thereof, or a prodrug ester thereof, and all stereoisomers thereof.

17. The pharmaceutical composition according to claim 1, wherein the DPP-IV inhibitor is a compound of formula (V) 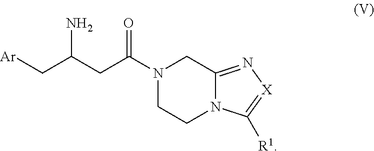
Ar is phenyl which is unsubstituted or substituted with 1-5 of R³, wherein R³ is independently selected from the group consisting of:
(1) halogen,
(2) C₁₋₆ alkyl, which is linear or branched and is unsubstituted or substituted with 1-5 halogens,
(3) OC₁₋₆ alkyl, which is linear or branched and is unsubstituted or substituted with 1-5 halogens, and
(4) CN;
X is selected from the group consisting of:
(1) N, and
(2) CR²;
R¹ and R² are independently selected from the group consisting of:
(1) hydrogen,
(2) CN,
(3) C₁₋₁₀ alkyl, which is linear or branched and which is unsubstituted or substituted with 1-5 halogens or phenyl, which is unsubstituted or substituted with 1-5 substituents independently selected from halogen, CN, OH, R⁴, OR⁴, NHSO₂R⁴, SO₂R⁴, CO₂H, and CO₂C₁₋₆alkyl, wherein the CO₂C₁₋₆ alkyl is linear or branched,
(4) phenyl which is unsubstituted or substituted with 1-5 substituents independently selected from halogen, CN, OH, R⁴, OR⁴, NHSO₂R⁴, SO₂R⁴, CO₂H, and CO₂C₁₋₆alkyl, wherein the CO₂C₁₋₆alkyl is linear or branched, and
(5) a 5- or 6-membered heterocycle which may be saturated or unsaturated comprising 1-4 heteroatoms independently selected from N, S and O, the heterocycle being unsubstituted or substituted with 1-3 substituents independently selected from oxo, OH, halogen, C₁₋₆alkyl, and OC₁₋₆alkyl, wherein the C₁₋₆alkyl and OC₁₋₆alkyl are linear or branched and optionally substituted with 1-5 halogens;
R⁴ is C₁₋₆alkyl, which is linear or branched and which is unsubstituted or substituted with 1-5 groups independently selected from halogen, CO₂H, and CO₂C₁₋₆alkyl, wherein the CO₂C₁₋₆alkyl is linear or branched;
and pharmaceutically acceptable salts thereof and individual diastereomers thereof.

18. The pharmaceutical composition according to claim 1, wherein the DPP-IV inhibitor is (2S)-1-{[2-(5-Methyl-2-phenyl-oxazol-4-yl)-ethylamino]-acetyl}-pyrrolidine-2-carbonitrile, or a pharmaceutically acceptable salt thereof.

19. The pharmaceutical composition according to claim 1, wherein the DPP-IV inhibitor is (2S)-1-{[1,1-Dimethyl-3-(4-pyridin-3-yl-imidazol-1-yl)-propylamino]-acetyl}-pyrrolidine-2-carbonitrile, or a pharmaceutically acceptable salt thereof.

20. The pharmaceutical composition according to claim 1, wherein the DPP-IV inhibitor is (S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one, or a pharmaceutically acceptable salt thereof.

21. The pharmaceutical composition according to claim 1, wherein the DPP-IV inhibitor is (S,S,S,S)-1-(2-Amino-9,10-dimethoxy-1,3,4,6,7,11b-hexahydro-2H-pyrido[2,1-a]isoquinolin-3-yl)-4-methyl-pyrrolidin-2-one, or a pharmaceutically acceptable salt thereof.

22. The pharmaceutical composition according to claim 1, wherein the DPP-IV inhibitor is (S)-1-[2-((5S,7S)-3-Hydroxy-adamantan-1-ylamino)-acetyl]-pyrrolidine-2-carbonitrile, or a pharmaceutically acceptable salt thereof.

23. The pharmaceutical composition according to claim 1, wherein the DPP-IV inhibitor is (1S,3S,5S)-2-[(S)-2-Amino-2-(3-hydroxy-adamantan-1-yl)-acetyl]-2-aza-bicyclo[3.1.0]hexane-3-carbonitrile, or a pharmaceutically acceptable salt thereof.

24. The pharmaceutical composition according to claim 1, wherein the DPP-IV inhibitor is (R)-3-Amino-1-(3-trifluoromethyl-5,6-dihydro-8H-[1,2,4]triazolo[4,3-a]pyrazin-7-yl)-4-(2,4,5-trifluoro-phenyl)-butan-1-one, or a pharmaceutically acceptable salts thereof.

25. The pharmaceutical composition according to claim 1, additionally comprising a DPP-IV inhibitor which is released in the stomach or upper gut.

26. The pharmaceutical composition according to claim 25, wherein 40 to 60% of the DPP-IV inhibitor is released in the stomach or upper gut and 40 to 60% of the DPP-IV inhibitor is released in the lower gastrointestinal tract.

27. The pharmaceutical composition according to claim 26, wherein the DPP-IV inhibitor is not released in the duodenum.

28. The pharmaceutical composition according to claim 25, wherein said pharmaceutical composition is a two layer tablet.

29. A method for the treatment of diseases associated with elevated blood glucose levels, comprising the step of administering a therapeutically effective amount of a pharmaceutical composition according to claim 1 to a human being or animal in need thereof.

30. The method according to claim 29, wherein said disease is type I diabetes mellitus, type II diabetes mellitus, diabetes secondary to pancreatic disease, diabetes related to steroid use, type III diabetes mellitus, hyperglycaemia, diabetic complications or insulin resistance.
